(19) United States
(12) Patent Application Publication
(10) Pub. No.: US 2012/0135927 A1
(43) Pub. Date: May 31, 2012

(54) BONE MORPHOGENETIC PROTEINS FOR THE TREATMENT OF INSULIN RESISTANCE

(75) Inventors: Yu-Hua Tseng, Newton, MA (US); C. Ronald Kahn, West Newton, MA (US)

(73) Assignee: JOSLIN DIABETES CENTER, INC., Boston, MA (US)

(21) Appl. No.: 13/321,542

(22) PCT Filed: May 21, 2010
(86) PCT No.: PCT/US10/35831

§ 371 Date: Feb. 15, 2012

Related U.S. Application Data
(62) Division of application No. 61/180,371, filed on May 21, 2009.

(51) Int. Cl. A61K 38/18; A61P 3/00

(52) U.S. Cl. 514/8.8

(57) ABSTRACT

The present disclosure relates to compositions and methods for treating insulin resistance and/or obesity in a cell and/or a subject.

### CLAIM OF PRIORITY

This application claims the benefit of U.S. Patent Application Ser. No. 61/180,371, filed on May 21, 2009, the entire contents of which are hereby incorporated by reference.

### FEDERALLY SPONSORED RESEARCH OR DEVELOPMENT

This invention was made with Government support under Grant Nos. DK070722 and DK077097 awarded by the National Institutes of Health. The Government has certain rights in the invention.

### TECHNICAL FIELD

This disclosure relates to compositions and methods for the treatment of insulin resistance and for the generation of brown adipocytes.

### BACKGROUND

Insulin resistance is when the body produces normal amounts of insulin that the body is not able to use properly. When a person suffers from insulin resistance, their muscles, fat and liver cells become less sensitive to the glucose-lowering effects of insulin. As a result, more insulin is required to promote glucose entry into cells. Initially, this demand for more insulin may be met by the pancreas. When the pancreas can no longer produce sufficient insulin, excess glucose builds up in the bloodstream. Many people with insulin resistance have high levels of both glucose and insulin circulating in their blood at the same time.

### SUMMARY

This disclosure provides, inter alia, methods for using bone morphogenic protein (BMP (e.g., BMP7)), and compositions comprising BMP agents (e.g., BMP7 agents), for treating insulin resistance. More specifically, this disclose provide compositions and methods that promote increased insulin sensitivity in vivo and in vitro. This disclosure also provides methods for using bone morphogenic protein (BMP (e.g., BMP7)), and compositions comprising BMP agents (e.g., BMP7 agents), for promoting (e.g., increasing) brown adipogenesis in insulin resistant cells in vivo and vitro. More particularly, the disclosure also provides compositions and methods for promoting the differentiation of a cell with a diminished ability to differentiate to a brown adipocyte (e.g., an insulin resistant cell), to a brown adipocyte. These differentiated cells can then be used, e.g., in cell therapy methods, e.g., to treat obesity and diabetes.

In some aspects, the disclosure provides methods of treating a condition associated with insulin resistance (e.g., insulin resistance or a condition causally linked insulin resistance) in a subject. These methods can include selecting a subject with insulin resistance (e.g., insulin resistance or a condition associated with insulin resistance), and administering a therapeutically effective amount of one or more BMP7 agents to the subject, thereby treating insulin resistance in the subject. Subjects with insulin resistance can include subjects with insulin resistance as defined in the field of endocrinology (e.g., subjects with a level of insulin sensitivity that is at or below the threshold level (e.g., 10%) of a sample of healthy individuals) or subjects diagnosed to have a disease with which insulin resistance is associated (e.g., causally linked), such as, for example, one or more of diabetes (e.g., type II diabetes), pre-diabetes, obesity, metabolic syndrome (syndrome X), cardiovascular disease (e.g., hypertension (e.g., up to 50% of patients with hypertension are estimated to have insulin resistance), arteriosclerosis (also known as atherosclerosis), reproductive abnormalities, polycystic ovary disease, hyperandrogenism, growth abnormalities, and fatty liver (steatosis). In some instances, insulin resistance can be assessed directly or indirectly.

In some embodiments, the BMP7 agent can be administered systemically or locally to a target cell, tissue, or organ. Such BMP7 agents can include any compound or composition that increases BMP7 expression, activity, and/or signaling in a target cell, tissue, or organ, and can include, for example: BMP7 polypeptides (e.g., a polypeptide with at least 95% identity to amino acids 293-431 of SEQ ID NO:5), BMP7 peptides or peptide mimetics, nucleic acid molecules encoding a BMP7 polypeptide (e.g., a BMP7 polypeptide with at least 95% identity to amino acids 293-431 of SEQ ID NO:5).

In some instances, the methods can include assessing or evaluating the level of insulin resistance in the subject before and after administration of a BMP7 agent, e.g., to determine the level success of the treatment, and/or to determine the duration of treatment. For example, a decrease in the level of insulin resistance post treatment, relative to the level of insulin resistance before treatment, can indicate a level of success. Treatment can then be continued or stopped. Similar assessments or evaluations can also be made at intervals following cessation of treatment. In some instance, evaluations of the level of insulin resistance can be made directly (e.g., by measuring insulin resistance or sensitivity), indirectly (e.g., by measuring glucose uptake or levels), and/or by assessing the level of a condition associated with insulin resistance, such as, e.g., one or more of diabetes (e.g., type II diabetes), pre-diabetes, obesity, metabolic syndrome (syndrome X), cardiovascular disease (e.g., hypertension (e.g., up to 50% of patients with hypertension are estimated to have insulin resistance), arteriosclerosis (also known as atherosclerosis), reproductive abnormalities, polycystic ovary disease, hyperandrogenism, growth abnormalities, and fatty liver (steatosis).

In some aspects, the disclosure provides methods for generating a brown adipocyte in vitro. Such methods can include, obtained (e.g., isolating (e.g., isolating from an insulin resistant subject) an insulin resistant cell (e.g., a cell that does not take up glucose in response to insulin or a cell with a diminished ability to take up glucose in response to insulin); and contacting (e.g., treating) the insulin resistant cell in vitro with an effective amount of a BMP7 agent under conditions and for a time sufficient for the insulin resistant cell to differentiate to a brown adipocyte, or genetically engineering the cell to express BMP-7. In some instances, cells useful in these methods can be, e.g., preadipocytes. Preadipocytes can be identified by detecting the expression (e.g., mRNA or protein expression) of one or more of PPARγ, C/EBPα, C/EBPβ, C/EBPδ, Glut4, aP2, FAS, and/or adiponectin. In some instances, brown adipocytes can be identified, and distinguished from preadipocytes, e.g., by detecting the expression (e.g., mRNA or protein) of one or more of UCP1, PRDM16, PGC-1a, PGC-1b, ERRa, Tfam, ATPase f1a1, and/or COX7a1.

In some embodiments, the methods can further include culturing the population of cells in a first cell growth medium comprising 0.1-100 nM insulin and 0.1-10 nM triiodothyronine (T3), 0.1-5 μM isobutylmethylxanthine (IBMX), 0.1-50 mM dexamethasone, and 0.01-10 mM indomethacin; and culturing the population of cells in a second cell growth medium comprising insulin and T3. In some embodiments, the methods can include contacting the insulin resistant cell with, for example, one or more of BMP-1, BMP-3, peroxisome proliferator-activated receptor gamma (PPARγ), Retinoid X receptor, alpha (RXRα), insulin, T3, a thiazolidinedione (TZD), vitamin A, retinoic acid, insulin, glucocorticoid or agonist thereof, Wingless-type (Wnt), Insulin-like Growth Factor-1 (IGF-1), Epidermal growth factor (EGF), Fibroblast growth factor (FGF), Transforming growth factor (TGF)-α, TGF-β, Tumor necrosis factor alpha (TNFα), Macrophage colony stimulating factor (MCSF), Vascular endothelial growth factor (VEGF) and Platelet-derived growth factor (PDGF).

In some embodiments, insulin resistant cells useful in these methods can include, for example, cells obtained (e.g., isolated) from skeletal muscle, prostate tissue, dermis tissue, tissue from the cardiovascular system, mammary gland tissue, liver tissue, neonatal skin, calvaria, bone marrow, intestinal tissue, adipose tissue, and adipose tissue deposits. In some embodiments, the insulin resistant cell is a Sca-1+ cell.

BMP7 agents useful in these methods can include, e.g., any compound or composition that increases BMP7 expression, activity, and/or signaling in a target cell, tissue, or organ, and can include, for example: BMP7 polypeptides (e.g., a polypeptide with at least 95% identity to amino acids 293-431 of SEQ ID NO:5), BMP7 peptides or peptide mimetics, nucleic acid molecules encoding a BMP7 polypeptide (e.g., a BMP7 polypeptide with at least 95% identity to amino acids 293-431 of SEQ ID NO:5).

In some embodiments, the insulin resistant cell can be cultured in a first cell growth medium containing 20 nM insulin and 1 nM triiodothyronine (T3), 0.5 μM isobutylmethylxanthine (IBMX), 5 mM dexamethasone, and 0.125 mM indomethacin.

In some embodiments, cells resulting from these methods (e.g., brown adipocytes) can be, e.g., administered to a subject either alone or in combination with a BMP7 agent.

In some aspects the disclosure includes a population of cells made by the methods disclosed herein and pharmaceutical compositions including a cell or a population of cells made by the methods in combination with and a pharmaceutically acceptable carrier. In some embodiments, such compositions can be administered to a subject, e.g., alone or in combination with a BMP7 agent.

In some aspects, the disclosure provides a population of cells made by the methods disclosed herein and pharmaceutical compositions including a cell or a population of cells made by the methods in a suitable cell delivery system that includes, e.g., a reservoir containing the cell or cells in a pharmaceutically acceptable carrier, and a delivery device in fluid contact with the reservoir.

In some embodiments, the present disclosure provides compositions and methods for increasing insulin sensitivity in a subject (e.g., a subject with insulin resistance). Such methods can include selecting a subject with insulin resistance (e.g., a subject that has been confirmed to have insulin resistance by one or more blood or laboratory tests (e.g., insulin tolerance test (ITT), a subject that a clinician believes has insulin resistance due to the diagnosis of a condition, by the clinician, that is associated with insulin resistance, a subject with a condition associated with insulin resistance, and/or a subject with one or more risk factors for developing insulin resistance), administering to the subject a compound that specifically increases BMP (e.g., BMP-7) expression, activity, and/or signaling in the subject (e.g., in insulin resistant cells in the subject), and optionally evaluating insulin resistance in the subject (e.g., before and/or after treatment).

In some embodiments, the present disclosure provides methods for increasing the number of cells with characteristics of brown adipose tissue (BAT) cells in a population of insulin resistant cells in vitro. These methods can include obtaining a population of insulin resistant cells; and contacting the population of cells in vitro with an effective amount of a compound that promotes increased expression of BMP7 (e.g., a BMP7 agent) for a time sufficient to increase the number of cells with characteristics of brown adipose tissue (BAT) cells in the population of cells; or genetically engineering the population of cells to express BMP-7 at a level sufficient to increase the number of cells with characteristics of BAT cells in the population of cells. In some aspects, the methods include implanting cells resulting from the methods disclosed herein in a subject (e.g., an autologous implant).

As used herein, “treatment” means any manner in which one or more of the symptoms of a disease or disorder are ameliorated or otherwise beneficially altered. As used herein, amelioration of the symptoms of a particular disease or disorder refers to any lessening, whether permanent or temporary, lasting or transient that can be attributed to or associated with treatment by the compositions and methods of the present invention.

The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome.

The term “subject” is used throughout the specification to describe an animal, human or non-human, to whom treatment according to the methods of the present invention is provided. Veterinary and non-veterinary applications are contemplated. The term includes, but is not limited to, birds and mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

In some embodiments, a subject can include a human or non human animal that would benefit therapeutically from treatment with the compositions and methods disclosed herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a diagram illustrating the experimental protocol followed in Example 1.

FIGS. 1B-1C are images of Petri dishes containing cultured C3H10T12 cells stained with Oil Red O at day 10 of the experimental protocol illustrated in FIG. 1A.

FIG. 1D is a diagram showing coordinated changes in several insulin signaling components after 3 days of BMP7 treatment. Numbers indicate the fold change of a given gene relative to control. Upregulated genes are marked with a cross. Downregulated genes are marked with a star. Data is also shown in Table 1.

FIG. 2A is an illustration showing the differentiation protocol used in Example 2.

FIG. 2B is an image of Petri dishes containing wild type of IRS-1KO cells stained with Oil Red O at day 10 of the experimental protocol illustrated in FIG. 2A.

FIG. 2C shows histograms showing the results of quantitative RT-PCR analysis of the expression of adipogenic markers at day 10 of the differentiation protocol shown in FIG. 2A. For Q-RT-PCRanalysis, data is presented as mean±SEM from a representative of 4 independent experiments with each performed in triplicates. The values for WT cells are set as 100 arbitrarily. P values or asterisks representing statistically significant differences between vehicle and BMP7-treated IRS-1KO cells are placed above the bars (* p<0.05, ** p<0.01, **** p<0.0001).

FIG. 2D shows histograms showing the results of quantitative RT-PCR analysis of the expression of brown fat selective genes at day 10 of the differentiation protocol shown in FIG. 2A.

FIG. 2E is an image of a Western blot showing analysis of general adipogenic and brown fat-specific marker proteins. A representative of 4 independent experiments is shown.

FIG. 3A shows histograms showing Q-RT-PCT analysis of adipogenic markers and inhibitors on day 3. V, Vehicle. Data presented as mean±SEM from a representative of 4 independent experiments with each performed in triplicates. The values for WT cells are set as 100 arbitrarily. Asterisks depict statistically significant differences between vehicle and BMP7-treated IRS-1KO cells. (* p<0.05, *** p<0.001).

FIG. 3B is an image of a Western blot showing analysis of early adipogenic inhibitor and marker proteins on day 3. A representative of 4 independent experiments is shown.

FIG. 4A is a bar graph showing Pref-1 mRNA expression in wild type (WT) and IRS1-KO cells overexpressing Smad1 and Smad4 (Smad1/4) or a dominant negative form of Smad4 (pCMV5-DPC4 (DPC4)) that were treated with vehicle (left side in each pair) or BMP-7 (right side column in each pair). mRNA were isolated and analyzed for Pref-1 expression by Q-RT-PCR. Data represents mean±SEM from a representative of 4 independent experiments with each performed in triplicates. The value for vehicle-treated vector-transfected cells is set as 100 arbitrarily. Asterisks depict statistically significant difference (* p<0.05, ** p<0.01).

FIGS. 4B-C are bar graphs showing the results of analysis of binding of Smad1 (4B) and Smad4 (4C) to the Pref-1 promoter, in wild type and IRS1-KO cells co-transfected with pGL(−201) or pGL(−201mut) along with pCS2-Flag-Smad1/pCS2-Flag-Smad4. Eighteen hours after transfection, cells were treated with vehicle or BMP7 for 1 hour.

FIG. 5A is a schematic of the Pref-1 promoter. The Smad binding elements (SBE) are shown.

FIG. 5B is an illustration showing the strategy used to map essential element(s) in Pref-1 promoter by sequential deletion.

FIG. 5C is an alignment of the putative SBE sequences in the pref-1 promoter from different species. The SBE9 sequences shared 100% identity among mice, rats and humans (i.e., GTCT), while the other SBEs exhibited a different degree of variation.

FIG. 5D is a histogram showing luciferase activity of reporter constructs containing the elements shown in 5B.

FIG. 5E is a histogram showing ChIP analysis of Smad1/4 binding to pref-1 promoter.

FIGS. 6A-6B are images of Petri dishes containing Oil Red O stained WT and IRS1-KO cells overexpressing BMP-7.

FIG. 6C is a histogram showing BMP7 overexpression in wild type and IRS-1 KO brown preadipocytes.

FIGS. 6D-6E are bar graphs showing Q-RT-PCR analysis of expression of adipogenic markers and brown-fat-selective genes in WT cells (C) and IRS-1KO cells (D) was analyzed by Q-RT-PCT. Data are presented as mean±SEM from a representative of 3 independent experiments with each performed in duplicates or triplicates. All the values for vector are set as 100 arbitrarily. The p values or asterisks depicting statistically significant differences between vehicle and BMP7 treatments were placed above the bars (* p<0.05, ** p<0.01, *** p<0.001).

FIGS. 7A-7B are images of Oil Red O stained control or Noggin expressing cells.

FIGS. 7C-7F are histograms showing gene expression levels in control or Noggin expressing cells.

FIG. 8A is a line graph showing insulin tolerance in control or BMP-7 treated DIO mice, as assessed using the insulin tolerance test (ITT).

FIG. 8B is a line graph showing glucose tolerance in control or BMP-7 treated DIO mice.

FIG. 8C is a photomicrograph showing sections of liver tissue from control (Ad-LacZ, top panels) and BMP-7 (Ad-BMP-7, lower panels) treated DIO mice.

### DETAILED DESCRIPTION

The present disclosure is based, at least in part, on the surprising discovery that BMP (e.g., BMP7) promotes increased insulin sensitivity, e.g., in insulin resistant cells, and rescues brown adipogenesis in insulin resistant cells. Accordingly, the disclosure provides compositions and methods for treating insulin resistance in vivo and in vitro using direct therapy (e.g., by administering BMP agents). The disclosure also provides cell therapy methods in which an insulin resistance preadipocyte that is differentiated to a brown adipocyte in vitro is transplanted into a subject, e.g., the subject from which the cell was derived.

### Compositions and Methods for Modulating BMP

The direct therapy and cell therapy methods disclosed herein can include modulating (e.g., increasing) BMP expression, activity, and/or signaling using a BMP agent. For example, the methods disclosed herein can require modulating (e.g., increasing) the expression, activity, and/or signaling of BMP (e.g., BMP7). Such can include a change in expression, activity, and/or signaling, such that the resulting expression, activity, and/or signaling is greater than the expression, activity, and/or signaling prior to treatment.

As used herein, “BMP-treated” refers to a cell or subject with an artificial level of BMP expression, activity, and/or signaling, e.g., BMP 2, -4, -5, -6, and/or -7 expression, activity, and/or signaling. “Artificial” means that the level of BMP signaling has been increased by direct human intervention.

In some instances, an increase in BMP expression, activity, and/or signaling can be promoted by: increasing the expression or activity of endogenous BMP or BMP receptors by administering pharmaceuticals or biologics that promote such a response; administering exogenous BMP or BMP receptors; or both. Such compositions are referred to collectively herein as BMP agents. BMP agents can include, but are not limited to, e.g., one or more of: (a) BMP2, -4, -5, -6, and/or -7 polypeptides or functional fragments or variants thereof (e.g., active (e.g., BMPR-I and/or BMPR-II activating) BMP2, -4, -5, -6, and/or -7 polypeptides or a functional fragments or analogs thereof (e.g., a mature BMP2, -4, -5, -6, and/or -7 polypeptide, e.g., a mature BMP2, -4, -5, -6, and/or -7 polypeptide described herein)); (b) peptides or protein agonists of a BMP2, -4, -5, -6, and/or -7 receptor, e.g., that increases the activity of a BMP receptor (e.g., BMPR-I and/or BMPR-II) either in the absence of BMP or by increasing or stabilizing binding of BMP2, -4, -5, -6, and/or -7 to its receptor); (c) small molecules or protein mimetics that mimic BMP2, -4, -5, -6, and/or -7 signaling activity (e.g., BMPR-I and/or BMPR-II binding activity, or SMAD phosphorylating activity); (d) small molecules that increase expression of one or more of BMP2, -4, -5, -6, and/or -7 (e.g., by binding to the promoter region of a BMP2, -4, -5, -6, and/or -7 gene); (e) antibodies or antibody fragments (e.g., a recombinant antibody or antibody fragment), e.g., antibodies or antibody fragments that bind (e.g., bind specifically) to a BMP receptor or antibodies and/or antibody fragments that stabilize or assist the binding of BMP2, -4, -5, -6, and/or -7 to a BMP2, -4, -5, -6, and/or -7 binding partner (e.g., a BMP2, -4, -5, -6, and/or -7 receptor); and that promotes biological activity of the receptor. Such antibodies or antibody fragments include, but are not limited to, for example, murine, chimeric, humanized, human, monoclonal, and polyclonal antibodies; antigen binding fragments of murine, chimeric, humanized, human, monoclonal, and polyclonal antibodies (e.g., monovalent fragments (e.g., Fab and ScFv) and engineered variants (e.g., diabodies, triabodies, minibodies, and single domain antibodies); and binding peptides containing an antigen binding portion of an BMP receptor-binding antibody (e.g., a complementarity domain region (CDR) (e.g., CDR3, CDR2, and/or CDR1); and/or (f) nucleic acids encoding a BMP2, -4, -5, -6, and/or -7 polypeptide or functional fragment or analog thereof. Such nucleic acids include genomic and cDNA sequences. BMP agents can also include BMP (polypeptides and nucleic acids) and functional equivalents of BMP (e.g., agents that target (e.g., specifically target) a selected BMP receptor (e.g., a BMP7 receptor) and evoke the same (e.g., substantially the same) biological effect produced upon binding of BMP to the receptor), such as, but not limited to, for example, pharmaceutical and biologics (including biosimilars and/or bioequivalents), such as, mimetics (e.g., protein mimetics), small molecules, antibodies and antibody fragments, and BMP receptor agonists.

BMP Polypeptides and Nucleic Acids

In some embodiments, a BMP agent can include a BMP2, -4, -5, -6, and/or -7 polypeptide or a nucleic acid molecule encoding BMP2, -4, -5, -6, and/or -7.

Polypeptides

BMP polypeptides can include, e.g., recombinant BMP polypeptides and chemically synthesized BMP polypeptides. BMP polypeptides (e.g., a mature BMP polypeptide) are themselves viable therapeutic compound because BMPs are small secreted proteins that are internalized into their target cells where they exert their activity. Although the human proteins are described herein, one of skill in the art will appreciate that when another species is the intended recipient of the treated cells, homologous proteins from that species can also be used, e.g., cow, pig, sheep, or goat. Such homologous proteins can be identified, e.g., using methods known in the art, e.g., searching available databases for homologs identified in the target species, e.g., the homologene database.

In some embodiments, a BMP polypeptide can be isolated or purified. Such polypeptides can be free (e.g., substantially free) of cellular material or other contaminating proteins from the cell or tissue source from which the protein is derived, or substantially free from chemical precursors or other chemicals when chemically synthesized. “Substantially free” means that the preparation of a selected protein has less than about 30%, (e.g., less than 20%, 10%, or 5%) by dry weight, of non-selected protein or of chemical precursors. Such a non-selected protein is also referred to herein as “contaminating protein,” When the isolated therapeutic proteins, peptides, or polypeptides are recombinantly produced, they can be substantially free of culture medium, i.e., culture medium represents less than about 20%, (e.g., less than about 10% or 5%) of the volume of the protein preparation.

BMP2

BMP2 is 396 amino acids in length, localized to chromosome 20p12 in human. The nucleotide and amino acid sequences of human BMP2 are disclosed in Wozney et al., Science 242(4885):1528-1534 (1988). BMP2 belongs to the transforming growth factor-beta (TGFβ) superfamily. Bone morphogenetic protein induces bone formation, and BMP2 is a candidate gene for the autosomal dominant disease of fibrodysplasia (myositis) ossificans progressive. Bone morphogenetic protein 2 regulates myogenesis through dosage-dependent PAX3 expression in pre-myogenic cells, and is expressed in mesoderm under SHM control through the SOX9.

The human BMP2 amino acid sequence is shown below Amino acids 38-268 are the TGFβ propeptide domain, and 291-396 are the TGFβ family N-terminal domain. Amino acids 283-396 are the mature peptide. The sequence is set forth in Wozney et al., Science 242:1528-1534 (1988).

(SEQ ID NO: 1)
MVAGTRCLLALLLPQVLLGGAAGLVPELGRRKFAAASSGRPSSQPSDEVL
SEFELRLLSMFGLKQRPTPSRDAVVPPYMLDLYRRHSGQPGSPAPDHRLE
RAASRANTVRSFHHEESLEELPETSGKTTRRFFFNLSSIPTEEFITSAEL
QVFREQMQDALGNNSSFHHRINIYEIIKPATANSKFPVTRLLDTRLVNQN
ASRWESFDVTPAVMRWTAQGHANHGFVVEVAHLEEKQGVSKRHVRISRSL
HQDEHSWSQIRPLLVTFGHDGKGHPLHKREKRQAKHKQRKRLKSSCKRHP
LYVDFSDVGWNDWIVAPPGYHAFYCHGECPFPLADHLNSTNHAIVQTLVN
SVNSKIPKACCVPTELSAISMLYLDENEKVVLKNYQDMVVEGCGCR

The mature form of BMP2 contains four potential N-linked glycosylation sites per polypeptide chain, and four potential disulfide bridges. See UniProt entry No. P12643; HomoloGene:926; GenBank Acc. Nos. NM_{—}001200.2 (mRNA) and NP_{—}001191.1 (protein).

In some embodiments, the BMP agent is a BMP2 polypeptide, e.g., human BMP2, e.g., a mature BMP2 polypeptide, e.g., a BMP2 polypeptide that includes amino acids 283-396 of SEQ ID NO:1.

BMP4

BMP4 induces cartilage and bone formation, and is important in mesoderm induction, tooth development, limb formation and fracture repair. The amino acid sequence of the BMP4 preproprotein is shown below Amino acids 41-276 are the TGFβ propeptide domain, and 302-408 are the TGFβ family N-terminal domain. Amino acids 293-408 are the mature peptide. The sequence is set forth in Wozney et al., Science 242:1528-1534 (1988).

(SEQ ID NO: 2)
MIPGNRMLMVVLLCQVLLGGASHASLIPETGKKKVAEIQGHAGGRRSGQS
HELLRDFEATLLQMFGLRRRPQPSKSAVIPDYMRDLYRLQSGEEEEEQIH
STGLEYPERPASRANTVRSFHHEEHLENIPGTSENSAFRFLFNLSSIPEN
EAISSAELRLFREQVDQGPDWERGFHRINIYEVMKPPAEVVPGHLITRLL
DTRLVHHNVTRWETFDVSPAVLRWTREKQPNYGLAIEVTHLHQTRTHQGQ
HVRISRSLPQGSGNWAQLRPLLVTFGHDGRGHALTRRRRAKRSPKHHSQR
ARKKNKNCRRHSLYVDFSDVGWNDWIVAPPGYQAFYCHGDCPFPLADHLN
STNHAIVQTLVNSVNSSIPKACCVPTELSAISMLYLDEYDKVVLKNYQEM
VVEGCGCR

The mature form of BMP4 contains four potential N-linked glycosylation sites per polypeptide chain. A variant exists in which V152 is an A. See UniProt Accession No. P 12644; HomoloGene:7247; GenBank Acc. Nos. NM_{—}001202.3 (mRNA, var. 1) and NP_{—}001193.2 (protein, var. 1); NM_{—}130850.2 (mRNA, var. 2) and NP 570911.2 (protein, var. 2). and NM_{—}130851.2 (mRNA, var. 3) and NP_{—}570912.2 (protein, var. 3).

In some embodiments, the BMP agent is a BMP4 polypeptide, e.g., human BMP4, e.g., a mature BMP4 polypeptide, e.g., a BMP4 polypeptide that includes amino acids 293-408 of SEQ ID NO:2.

BMP5

The BMP5 preproprotein is a 454 amino acid protein, as shown below. BMP5 induces cartilage and bone formation. The amino acid sequence of BMP5 is set forth in Celeste et al., Proc. Natl. Acad. Sci. U.S.A., 87, 9843-9847, 1990.

(SEQ ID NO: 3)
MHLTVFLLKGIVGFLWSCWVLVGYAKGGLGDNHVHSSFIYRRLRNHERRE
IQREILSILGLPHRPRPFSPGKQASSAPLFMLDLYNAMTNEENPEESEYS
VRASLAEETRGARKGYPASPNGYPRRIQLSRTTPLTTQSPPLASLHDTNF
LNDADMVMSFVNLVERDKDFSHQRRHYKEFRFDLTQIPHGEAVTAAEFRI
YKDRSNNRFENETIKISIYQIIKEYTNRDADLFLLDTRKAQALDVGWLVF
DITVTSNHWVINPQNNLGLQLCAETGDGRSINVKSAGLVGRQGPQSKQPF
MVAFFKASEVLLRSVRAANKRKNQNRNKSSSHQDSSRMSSVGDYNTSEQK
QACKKHELYVSFRDLGWQDWITAPEGYAAFYCDGECSFPLNAHMNATNHA
IVQTLVHLMFPDHVPKPCCAPTKLNAISVLYFDDSSNVILKKYRNMVVRS
CGCH

The mature BMP5 protein is believed to be amino acids 323-454 of SEQ ID NO:3, and has four potential N-linked glycosylation sites per polypeptide chain, and four potential disulfide bridges. See UniProt Accession Nos. P22003; Q9H547; or Q9NTM5; HomoloGene:22412; GenBank Acc. Nos. NM_{—}021073.2 (mRNA) and NP_{—}066551.1 (protein).

In some embodiments, the BMP agent is a BMP5 polypeptide, e.g., human BMP5, e.g., a mature BMP5 polypeptide, e.g., a BMP4 polypeptide that includes amino acids 323-454 of SEQ ID NO:3.

BMP6

BMP6 is an autocrine stimulator of chondrocyte differentiation, and is involved in the development of embryonic neural, and urinary systems, as well as growth and differentiation of liver and keratinocytes. BMP6 knockout mice are viable and show a slight delay in ossification of the sternum. BMP6 (precursor) is a 57 kD protein, 513 amino acids in length, localized to chromosome 6p24 in human. The nucleotide and amino acid sequence of human BMP6 is disclosed in U.S. Pat. No. 5,187,076. BMP6 is predicted to be synthesized as a precursor molecule which is cleaved to yield a 132 amino acid mature polypeptide with a calculated molecular weight of approximately 15 Kd. The mature form of BMP6 contains three potential N-linked glycosylation sites per polypeptide chain. The active BMP6 protein molecule is likely a dimer. Processing of BMP6 into the mature form involves dimerization and removal of the N-terminal region in a manner analogous to the processing of the related protein TGFβ (Gentry et al., Molec. Cell. Biol. 8:4162 (1988); Dernyck et al., Nature 316:701 (1985)). The human BMP6 precursor is shown below. The mature polypeptide is believed to include amino acids 374-513 of SEQ ID NO:4. Other active BMP6 polypeptides include polypeptides including amino acids 382-513, 388-513 and 412-513 of SEQ ID NO:4.

(SEQ ID NO: 4)
MPGLGRRAQWLCWWWGLLCSCCGPPPLRPPLPAAAAAAAGGQLLGDGGSP
GRTEQPPPSPQSSSGFLYRRLKTQEKREMQKEILSVLGLPHRPRPLHGLQ
QPQPPALRQQEEQQQQQQLPRGEPPPGRLKSAPLFMLDLYNALSADNDED
GASEGERQQSWPHEAASSSQRRQPPPGAAHPLNRKSLLAPGSGSGGASPL
TSAQDSAFLNDADMVMSFVNLVEYDKEFSPRQRHHKEFKFNLSQIPEGEV
VTAAEFRIYKDCVMGSFKNQTFLISIYQVLQEHQHRDSDLFLLDTRVVWA
SEEGWLEFDITATSNLWVVTPQHNMGLQLSVVTRDGVHVHPRAAGLVGRD
GPYDKQPFMVAFFKVSEVHVRTTRSASSRRRQQSRNRSTQSQDVARVSSA
SDYNSSELKTACRKHELYVSFQDLGWQDWIIAPKGYAANYCDGECSFPLN
AHMNATNHAIVQTLVHLMNPEYVPKPCCAPTKLNAISVLYFDDNSNVILK
KYRNMVVRACGCH

The human BMP6 promoter has been characterized (See Tamada et al., Biochim Biophys Acta. 1998, 1395(3):247-51), and can be used in methods described herein. See UniProt Accession No. P22004; HomoloGene:1300; GenBank Acc. Nos. NM_{—}001718.4 (mRNA) and NP_{—}001709.1 (protein).

Administration, antisense treatment, and quantitation of BMP6 are described in Boden et al. (Endocrinology Vol. 138, No. 7 2820-2828).

In some embodiments, the BMP agent is a BMP6 polypeptide, e.g., human BMP6, e.g., a mature BMP6 polypeptide, e.g., a BMP6 polypeptide that includes amino acids 374-513 of SEQ ID NO:4, amino acids 382-513 of SEQ ID NO:4, amino acids 388-513 of SEQ ID NO:4, or amino acids 412-513 of SEQ ID NO:4.

BMP7

BMP7 also belongs to the TGFβ superfamily. It induces cartilage and bone formation, and may be the osteoinductive factor responsible for the phenomenon of epithelial osteogenesis. BMP7 plays a role in calcium regulation and bone homeostasis, and in the regulation of anti-inflammatory response in the adult gut tissue. The sequence of BMP7 is shown below:

(SEQ ID NO: 5)
MHVRSLRAAAPHSFVALWAPLFLLRSALADFSLDNEVHSSFIHRRLRSQE
RREMQREILSILGLPHRPRPHLQGKHNSAPMFMLDLYNAMAVEEGGGPGG
QGFSYPYKAVFSTQGPPLASLQDSHFLTDADMVMSFVNLVEHDKEFFHPR
YHHREFRFDLSKIPEGEAVTAAEFRIYKDYIRERFDNETFRISVYQVLQE
HLGRESDLFLLDSRTLWASEEGWLVEDITATSNHWVVNPRHNLGLQLSVE
TLDGQSINPKLAGLIGRHGPQNKQPFMVAFFKATEVHFRSIRSTGSKQRS
QNRSKTPKNQEALRMANVAENSSSDQRQACKKHELYVSFRDLGWQDWIIA
PEGYAAYYCEGECAFPLNSYMNATNHAIVQTLVHFINPETVPKPCCAPTQ
LNAISVLYFDDSSNVILKKYRNMVVRACGCH

Amino acids 1-29 are a potential signal sequence; 30-431 are the prepropeptide, and 293-431 are the mature protein. The mature form of BMP7 contains four potential N-linked glycosylation sites per polypeptide chain, and four potential disulfide bridges. See UniProt Accession No. P18075; HomoloGene:20410; GenBank Acc. Nos. NM_{—}001719.2 (mRNA) and NP_{—}001710.1 (protein).

In some embodiments, the BMP agent is a BMP7 polypeptide, e.g., human BMP7, e.g., a mature BMP7 polypeptide, e.g., a BMP7 polypeptide that includes amino acids 293-431 of SEQ ID NO:5.

In some embodiments, BMP polypeptides can be at least 50%, 60%, 70%, 80%, 85%, 90%, 95%, 98%, 99%, or 100% homologous to a BMP sequence known in the art or described herein, e.g., SEQ ID NO:1, 2, 3, 4, and/or 5.

To determine the percent identity of two amino acid sequences, or of two nucleic acid sequences, such as those nucleic acid sequences disclosed below, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes is 100% of the length of the reference sequence (e.g., the full length). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The determination of percent identity between two amino acid sequences could be accomplished using the BLAST 2.0 program. Sequence comparison is performed using an ungapped alignment and using the default parameters (Blossom 62 matrix, gap existence cost of 11, per residue gapped cost of 1, and a lambda ratio of 0.85). The mathematical algorithm used in BLAST programs is described in Altschul et al. (Nucleic Acids Res. 25:3389-3402, 1997). Useful BMP encoding polypeptide sequences or polypeptide fragments can have up to about 20 (e.g., up to about 10, 5, or 3) amino acid deletions, additions, or substitutions, such as conservative substitutions, to be useful for the compositions and methods described herein. Conservative amino acid substitutions are known in the art.

In some embodiments, a BMP polypeptide can be modified, e.g., to increase one or more of the activity, stability, binding activity, and/or binding specificity of the polypeptide. For example, modifications can be made to a polypeptide that result in pharmacokinetic properties of the protein which are desirable for use in protein therapy. For example, such modifications can result in an increase in cellular uptake, circulatory half-life, rate of clearance and reduced immunogenicity. Several art-recognized approaches are known that are useful to optimize the therapeutic activity of a protein compound, e.g., a compound described herein such as a BMP2, -4, -5, -6, and/or -7 polypeptide.

For recombinant proteins, the choice of expression system can influence pharmacokinetic characteristics. Differences between expression systems in post-translational processing can lead to recombinant proteins of varying molecular size and charge, which can affect, for example, cellular uptake, circulatory half-life, rate of clearance and immunogenicity. The pharmacokinetic properties of the protein may be optimized by the appropriate selection of an expression system, such as selection of a bacterial, viral, or mammalian expression system. Exemplary mammalian cell lines useful in expression systems for therapeutic proteins are Chinese hamster ovary, (CHO) cells, the monkey COS-1 cell line and the CV-1 cell line.

A protein can be chemically altered to enhance the pharmacokinetic properties while maintaining activity. The protein can be covalently linked to a variety of moieties, altering the molecular size and charge of the protein and consequently its pharmacokinetic characteristics. The moieties are preferably non-toxic and biocompatible. In some embodiments, polyethylene glycol (PEG) can be covalently attached to the protein (PEGylation). See, e.g., Poly(ethylene glycol): Chemistry and Biological Applications, Harris and Zalipsky, eds., ACS Symposium Series, No. 680, 1997; Harris et al., Clinical Pharmacokinetics 40:7, 485-563 (2001)). In another embodiment, the protein can be similarly linked to oxidized dextrans via an amino group. (See Sheffield, Current Drug Targets—Cardiovas. and Haemat. Dis. 1:1, 1-22 (2001)).

Furthermore, the protein compounds can be chemically linked to another protein. The protein can be cross-linked carrier protein to form a larger molecular weight complex with improved cellular uptake. In some embodiments, the carrier protein can be a serum protein, such as albumin. The protein can be attached to one or more albumin molecules via a bifunctional cross-linking compound. The cross-linking compound may be homo- or heterofunctional. In another embodiment, the protein can cross-link with itself to form a homodimer, trimer, or higher analog. Again, either heterobifunctional or homobifunctional cross-linking compounds can be used to form the dimers or trimers. (See Stykowski et al., Proc. Natl. Acad. Sci. USA, 95, 1184-1188 (1998)).

Nucleic Acids

In some embodiments, the BMP agent can be a BMP nucleic acid that enhances BMP expression, activity, and/or signaling as described herein, which can include, e.g., a BMP nucleic acid, e.g., a BMP2, -4, -5, -6, and/or -7 encoding nucleic acid sequence or a biologically active fragment or analog thereof (e.g., a nucleic acid encoding one or more of SEQ ID NOs: 1-5 or a nucleic acid encoding a polypeptide with a sequence with at least 50%, 60%, 70%, 80%, 85%, 90%, 95%, 98%, 99%, or 100% homology to one or more of SEQ ID NOs: 1-5), and any of: a promoter sequence, e.g., a promoter sequence from a BMP2, -4, -5, -6, and/or -7 gene or from another gene; an enhancer sequence, e.g., 5′ untranslated region (UTR), e.g., a 5′ UTR from a BMP2, -4, -5, -6, and/or -7 gene or from another gene, a 3′ UTR, e.g., a 3′ UTR from a BMP2, -4, -5, -6, and/or -7 gene or from another gene; a polyadenylation site; an insulator sequence; or another sequence that enhances the expression of BMP2, -4, -5, -6, and/or -7.

In another embodiment, the level of BMP2, -4, -5, -6, and/or -7 protein is increased by increasing the level of expression of an endogenous BMP2, -4, -5, -6, and/or -7 gene, e.g., by increasing transcription of the BMP2, -4, -5, -6, and/or -7 gene or increasing BMP2, -4, -5, -6, and/or -7 mRNA stability. In some embodiments, transcription of the BMP2, -4, -5, -6, and/or -7 gene is increased by: altering the regulatory sequence of the endogenous BMP2, -4, -5, -6, and/or -7 gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the BMP2, -4, -5, -6, and/or -7 gene to be transcribed more efficiently. In some embodiments, the nucleic acid encodes or increases transcription of BMP7.

The nucleic acids described herein, e.g., a nucleic acid encoding a BMP2, -4, -5, -6, and/or -7 polypeptide as described herein, can be incorporated into a gene construct. The methods described herein can use such expression vectors for in vitro transfection and expression of a BMP2, -4, -5, -6, and/or -7 polypeptide described herein in particular cell types, e.g., stem cells, e.g., pluripotent mesenchymal stem cells. Expression constructs of such components can be administered in any biologically effective carrier, e.g., any formulation or composition capable of effectively delivering the component gene to cells in vivo. Approaches include insertion of a subject gene in viral vectors including recombinant retroviruses, adenovirus, adeno-associated virus, and herpes simplex virus-1, or recombinant bacterial or eukaryotic plasmids.

Viral vectors transfect cells directly, and infection of cells with a viral vector generally has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid. Retroviral vectors, adenovirus-derived vectors, and adeno-associated virus vectors can also be used as a recombinant gene delivery system for the transfer of exogenous genes. These vectors provide efficient delivery of genes into cells, and the transferred nucleic acids are generally stably integrated into the chromosomal DNA of the host. Protocols for producing recombinant retroviruses and for infecting cells in vitro or in vivo with such viruses can be found in Current Protocols in Molecular Biology, Ausubel et al. (eds.) Greene Publishing Associates, (1989), Sections 9.10-9.14, and other standard laboratory manuals.

Non-viral methods can also be employed to cause expression of an nucleic acid compound described herein (e.g., a BMP2, -4, -5, -6, and/or -7 polypeptide encoding nucleic acid) into a cell. Most nonviral methods of gene transfer rely on normal mechanisms used by mammalian cells for the uptake and intracellular transport of macromolecules. In some embodiments, non-viral gene delivery systems of the present invention rely on endocytic pathways for the uptake of the subject gene by the targeted cell. Plasmid DNA can be delivered with the help of, for example, cationic liposomes (e.g., LIPOFECTIN™) or derivatized (e.g., antibody conjugated), polylysine conjugates, gramicidin S, artificial viral envelopes or other such intracellular carriers, as well as direct injection of the gene construct or CaPO₄ precipitation. Other embodiments include plasmid injection systems such as are described in Meuli et al., J. Invest. Dermatol. 116(1):131-135 (2001); Cohen et al., Gene Ther 7(22):1896-905 (2000); or Tam et al., Gene Ther. 7(21):1867-74 (2000).

The nucleic acids described herein can be incorporated into a gene construct that facilitates administration and uptake of the nucleic acid by a selected cell or tissue type. The invention features expression vectors for in vivo transfection and expression of a BMP2, -4, -5, -6, and/or -7 polypeptide described herein in particular cell types. Expression constructs of such components may be administered in any biologically effective carrier, e.g. any formulation or composition capable of effectively delivering the component gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (e.g., LIPOFECTIN™) or derivatized (e.g. antibody conjugated), polylysine conjugates, gramicidin S, artificial viral envelopes or other such intracellular carriers, as well as direct injection of the gene construct or CaPO₄ precipitation carried out in vivo.

One approach for in vivo introduction of nucleic acid into a cell is by use of a viral vector containing nucleic acid, e.g. a cDNA, encoding an alternative pathway component described herein. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271-78 (1990)). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel et al. (eds.) Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include *Crip, *Cre, *2 and *Am. Retroviruses have been used to introduce a variety of genes into many different cell types, including epithelial cells, in vitro and/or in vivo (see for example Eglitis, et al., Science 230:1395-1398 (1985); Danos and Mulligan, Proc. Natl. Acad. Sci. USA 85:6460-6464 (1988); Wilson et al., Proc. Natl. Acad. Sci. USA 85:3014-3018 (1988); Armentano et al., Proc. Natl. Acad. Sci. USA 87:6141-6145 (1990); Huber et al., Proc. Natl. Acad. Sci. USA 88:8039-8043 (1991); Ferry et al., Proc. Natl. Acad. Sci. USA 88:8377-8381 (1991); Chowdhury et al., Science 254:1802-1805 (1991); van Beusechem et al., Proc. Natl. Acad. Sci. USA 89:7640-7644 (1992); Kay et al., Human Gene Therapy 3:641-647 (1992); Dai et al., Proc. Natl. Acad. Sci. USA 89:10892-10895 (1992); Hwu et al., J. Immunol. 150:4104-4115 (1993); U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting non-dividing cells and can be used to infect a wide variety of cell types, including epithelial cells (Rosenfeld et al. (1992), supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. (1998), supra; Haj-Ahmand and Graham, J. Virol. 57:267 (1986)).

Yet another viral vector system useful for delivery of the subject gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129 (1992)). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989)). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993)).

In addition to viral transfer methods, such as those illustrated above, non-viral methods can also be employed to cause expression of an nucleic acid agent described herein (e.g., a BMP2, -4, -5, -6, and/or -7 polypeptide encoding nucleic acid) in the tissue of a subject. Most nonviral methods of gene transfer rely on normal mechanisms used by mammalian cells for the uptake and intracellular transport of macromolecules. In some embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther 7(22):1896-905 (2000); or Tam et al., Gene Ther. 7(21):1867-74 (2000).

In a representative embodiment, a gene encoding an alternative pathway component described herein can be entrapped in liposomes bearing positive charges on their surface (e.g., lipofectins) and (optionally) which are tagged with antibodies against cell surface antigens of the target tissue (Mizuno et al., No Shinkei Geka 20:547-551 (1992); PCT publication WO91/06309; Japanese patent application 1047381; and European patent publication EP-A-43075).

In clinical settings, the gene delivery systems for the therapeutic gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al., PNAS 91: 3054-3057 (1994)).

In some embodiments, the BMP nucleic acid can be engineered to target neurons or optimally express either mRNA and/or the protein encoded thereby in the brain (e.g., in the hypothalamus).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

### Peptide Mimetics

In some embodiments, the BMP agent is a peptide mimetic (e.g., either a peptide or nonpeptide peptide mimetic). Synthesis of nonpeptide compounds that mimic peptide sequences is known in the art. Nonpeptide compounds that mimic, for example, BMP7, are specifically contemplated by the present invention. Such peptide mimetics include BMP peptides that can be modified according to methods known in the art for producing peptidomimetics. See, e.g., Kazmierski, W. M., ed., Peptidomimetics Protocols, Human Press (Totowa N.J. 1998); Goodman et al., eds., Houben-Weyl Methods of Organic Chemistry: Synthesis of Peptides and Peptidomimetics, Thiele Verlag (New York 2003); and Mayo et al., J. Biol. Chem., 278:45746, 2003. In some cases, these modified peptidomimetic versions of the peptides and fragments disclosed herein exhibit enhanced stability in vivo, relative to the non-peptidomimetic peptides. Methods for creating a peptidomimetic include substituting one or more, e.g., all, of the amino acids in a peptide sequence with D-amino acid enantiomers. Such sequences are referred to herein as “retro” sequences. In another method, the N-terminal to C-terminal order of the amino acid residues is reversed, such that the order of amino acid residues from the N-terminus to the C-terminus of the original peptide becomes the order of amino acid residues from the C-terminus to the N-terminus in the modified peptidomimetic. Such sequences can be referred to as “inverso” sequences. Peptidomimetics can be both the retro and inverso versions, i.e., the “retro-inverso” version of a peptide disclosed herein. The new peptidomimetics can be composed of D-amino acids arranged so that the order of amino acid residues from the N-terminus to the C-terminus in the peptidomimetic corresponds to the order of amino acid residues from the C-terminus to the N-terminus in the original peptide.

Other methods for making a peptidomimetics include replacing one or more amino acid residues in a peptide with a chemically distinct but recognized functional analog of the amino acid, i.e., an artificial amino acid analog. Artificial amino acid analogs include β-amino acids, β-substituted β-amino acids (“β³-amino acids”), phosphorous analogs of amino acids, such as α-amino phosphonic acids and α-amino phosphinic acids, and amino acids having non-peptide linkages. Artificial amino acids can be used to create peptidomimetics, such as peptoid oligomers (e.g., peptoid amide or ester analogues), β-peptides, cyclic peptides, oligourea or oligocarbamate peptides; or heterocyclic ring molecules. These sequences can be modified, e.g., by biotinylation of the amino terminus and amidation of the carboxy terminus

### Additional BMP Agents

In some embodiments, a BMP agent can include modulators of TDF-like receptors, e.g., as disclosed in U.S. Pat. No. 7,482,329, and U.S. Publication No. 2010/0015150 (e.g., compounds termed “Thrasos compounds that bind to ALK3”), which is incorporated by reference herein in its entirety. In some embodiments, a BMP agent can include peptides with the amino acid sequences shown below:

| | |
|---|---|
| CIVNSSDDFLCKKYRS | (SEQ ID NO: 6) |
| CYFNDSSQVLCKRYRS | (SEQ ID NO: 7) |

or peptide mimetics thereof, e.g., as described herein.

In some embodiments, a BMP agent can include the BMP7 mimetic AA123 (Thrasos Inc., Hopkinton, Ma). Similar to BMP7, AA123 binds specifically to immobilized extracellular domains (ECD) of ALK3 and BMPR-II and promotes SMAD01 translocation. Unlike BMP7, AA123 does not bind to ALK6.

In some embodiments, a BMP agent can include BMP2 mimetics and other proteins that increase or enhance BMP-2 activity such as, e.g., LIM mineralization protein-1 (LMP-1) and/or SVAK-3 as disclosed by Okada et al. (Cell. Biochem. Funct., 27:526-534 (2009)); and B2A2, as disclosed by Lin et al. (Journal of Bone and Mineral Res., 20:693-703 (2005)).

Any of the peptides described herein, including the variant forms described herein, can further include a heterologous polypeptide. The heterologous polypeptide can be a polypeptide that increases the circulating half-life of the peptide to which it is attached (e.g., fused, as in a fusion protein). The heterologous polypeptide can be an albumin (e.g., a human serum albumin or a portion thereof) or a portion of an immunoglobulin (e.g., the Fc region of an IgG). In some instances, the heterologous peptide can be a polypeptide that increases trafficking of the peptide to which it is attached across a cell membrane and/or to a target cell and/or tissue. Such methods are described in the art (see, e.g., Begley, J. Pharm. Pharmacol., 48:136-146 (1996)).

The present disclosure also contemplates synthetic mimicking compounds. As is known in the art, peptides can be synthesized by linking an amino group to a carboxyl group that has been activated by reaction with a coupling agent, such as dicyclohexylcarbodiimide (DCC). The attack of a free amino group on the activated carboxyl leads to the formation of a peptide bond and the release of dicyclohexylurea. It can be necessary to protect potentially reactive groups other than the amino and carboxyl groups intended to react. For example, the (α-amino group of the component containing the activated carboxyl group can be blocked with a tertbutyloxycarbonyl group. This protecting group can be subsequently removed by exposing the peptide to dilute acid, which leaves peptide bonds intact. With this method, peptides can be readily synthesized by a solid phase method by adding amino acids stepwise to a growing peptide chain that is linked to an insoluble matrix, such as polystyrene beads. The carboxyl-terminal amino acid (with an amino protecting group) of the desired peptide sequence is first anchored to the polystyrene beads. The protecting group of the amino acid is then removed. The next amino acid (with the protecting group) is added with the coupling agent. This is followed by a washing cycle. The cycle is repeated as necessary.

In some embodiments, the mimetics of the present disclosure are peptides having sequence homology to a herein-described BMP peptide. These mimetics include, but are not limited to, peptides in which L-amino acids are replaced by their D-isomers. One common methodology for evaluating sequence homology, and more importantly statistically significant similarities, is to use a Monte Carlo analysis using an algorithm written by Lipman and Pearson to obtain a Z value. According to this analysis, a Z value greater than 6 indicates probable significance, and a Z value greater than 10 is considered to be statistically significant (Pearson and Lipman, Proc. Natl. Acad. Sci. (USA), 85:2444-2448, 1988; Lipman and Pearson, Science, 227:1435-1441, 1985. More generally, the peptide ligands described herein and the mimetics described above can be synthesized using any known methods, including tea-bag methodology or solid phase peptide synthesis procedures described by Merrifield et al., Biochemistry, 21:5020-5031, 1982; Houghten Wellings, Proc. Natl. Acad. Sci. (USA), 82:5131-5135, 1985; Atherton, Methods in Enzymology, 289:44-66, 1997, or Guy and Fields, Methods in Enzymology, 289:67-83, 1997, or using a commercially available automated synthesizer.

### Pharmaceutical Formulations

The BMP agent can be incorporated into pharmaceutical compositions suitable for administration to a subject, e.g., a human. In some instances, the pharmaceutical formulation can be optimized for systemic administration or local administration (e.g., administration to a specific site, such as, for example, a fat depot, or another target organ (e.g., pancreas), tissue, or cell). Such compositions typically include one or more of the BMP agents disclosed herein and a pharmaceutically acceptable carrier. A pharmaceutically acceptable carrier can include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions. Such supplementary active compounds can include, but are not limited to, e.g., peroxisome proliferator-activated receptor gamma (PPAR), Retinoid X receptor, alpha (RXR), insulin, T3, a thiazolidinedione (TZD), retinoic acid, another BMP (e.g., the BMP being used and one or more of BMP 1, 2, 3, 4, 5, 6, or 7), vitamin A, retinoic acid, insulin, glucocorticoid or agonist thereof, Wingless-type (Wnt), e.g., Wnt-1, Insulin-like Growth Factor-1 (IGF-1), or other growth factor, e.g., Epidermal growth factor (EGF), Fibroblast growth factor (FGF), Transforming growth factor (TGF)-13, TGF-γ, Tumor necrosis factor alpha (TNFα), Macrophage colony stimulating factor (MCSF), Vascular endothelial growth factor (VEGF) and/or Platelet-derived growth factor (PDGF).

A pharmaceutical composition can be formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an agent described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL™ (sodium carboxymethyl starch), or corn starch; a lubricant such as magnesium stearate or STEROTES™; a glidant such as colloidal silicon dioxide; a sweetening agent such as sucrose or saccharin; or a flavoring agent such as peppermint, methyl salicylate, or orange flavoring.

For transmucosal or transdermal administration, penetrants appropriate to the barrier to be permeated can be included. Such penetrants are generally known, and include, for example, for transmucosal administration, detergents, bile salts, and fusidic acid derivatives. In addition, transdermal compositions can be formulated into ointments, salves, gels, or creams as generally known in the art

In some embodiments, the BMP agent can be encapsulated or can be contained in a matrix or carrier. The agent can be provided in a matrix capable of delivering the agent to the chosen site.

Matrices can provide slow release of the agent and provide proper presentation and appropriate environment for cellular infiltration. Matrices can be formed of materials presently in use for other implanted medical applications. The choice of matrix material is based on any one or more of: biocompatibility, biodegradability, mechanical properties, cosmetic appearance and interface properties. One example is a collagen matrix. Carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In some embodiments, the pharmaceutical composition can be included in a container, pack, or dispenser together with instructions for administration, e.g., in a kit.

Effective Dose

Toxicity and therapeutic efficacy of the compounds and pharmaceutical compositions described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Polypeptides or other compounds that exhibit large therapeutic indices are preferred.

Data obtained from cell culture assays and further animal studies can be used in formulating a range of dosage for use in humans. The dosage of such compounds lies preferably within a range of circulating concentrations that include the ED₅₀ with little or no toxicity, and with little or no adverse effect on a human's ability to hear. The dosage may vary within this range depending upon the dosage form employed and the route of administration utilized. For any compound used in the methods described herein, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Exemplary dosage amounts of a differentiation agent are at least from about 0.01 to 3000 mg per day, e.g., at least about 0.00001, 0.0001, 0.001, 0.01, 0.1, 1, 2, 5, 10, 25, 50, 100, 200, 500, 1000, 2000, or 3000 mg per kg per day, or more.

The formulations and routes of administration can be tailored to the disease or disorder being treated, and for the specific human being treated. A subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

Generally the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., Chapter 27 In: Remington's Pharmaceutical Sciences, 18th Ed., Gennaro, ed., Mack Publishing Co., Easton, Pa., 1990). Generally, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., Chapter 3, In: Goodman & Gilman's “The Pharmacological Basis of Therapeutics”, 9th Ed., Hardman et al., eds., McGraw-Hill, New York, N.Y., 1996).

### Methods for Treating Insulin Resistance In Vivo

In some embodiments, the present disclosure provides methods for treating insulin resistance in a subject by: selecting a subject; and administering to the subject a BMP agent (e.g., a BMP7 agent) under conditions and for a time suitable for a decrease in insulin resistance to occur in the subject. The methods can also include optionally evaluating insulin resistance in the subject, e.g., before, during, and/or after treatment, e.g., using any of the methods disclosed in the section entitled “subject selection” below.

In some embodiments, the methods include administering the compound in combination with a second treatment, e.g., a second treatment for obesity or an obesity related disorder, e.g., type II diabetes. For example, the second treatment can be insulin, orlistat, phendimetrazine, and/or phentermine.

The methods can optionally further include: repeating administration of the one or more BMP agents, for example, for a period sufficient to yield a decrease in insulin resistance in the subject. For example, treatment can continue until at least a 1%, 2%, 5%, 10%, 20%, 30%, 40%, 50%, 75%, 90%, 95%, 99%, or 100% decrease in insulin resistance, relative to the level of insulin resistance present in the subject prior to treatment, occurs in the subject. Accordingly, in some embodiments, the methods can include, at least, (1) evaluating insulin resistance in the subject prior to treatment, e.g., to determine the level of insulin resistance in the subject prior to treatment; and, optionally, (2) evaluating insulin resistance in the subject post treatment and/or at the conclusion of treatment, e.g., to determine the level of insulin resistance in the subject post treatment or at the conclusion of treatment. In some embodiments, post treatment evaluations can be performed days, weeks, or years after treatment. In some embodiments, the insulin level of a subject is evaluated at regular or fixed intervals post treatments (e.g., annually).

Alternatively or in addition, administration can be repeated until an improvement in a condition associated with insulin resistance occurs, e.g., until a decrease in the body weight of the subject occurs (weight can be assessed by weighing the subject). In some embodiments, treatment can be continued for a period prescribed by a health care professional, for a period elected by the subject, or for a period sufficient to reduce the subject's BMI; and/or monitoring food intake by the subject, each of which can be performed before and after administration of the one or more BMPs and/or before and after the subject has completed treatment (e.g., once no further administrations of one or more BMP agents are required). In some embodiments, treatment can be continued until a subject's BMI is below 30. In some embodiments, treatment can be continued until a subject's BMI is below 25.

In some embodiments, these direct therapy methods can be combined with the cell therapy methods disclosed below. For example, one or more BMP agents can be administered to a subject in combination with (e.g., simultaneously with or during the same course of treatment) a brown adipocyte generated using the methods described herein.

### Subject Selection

In some embodiments, the methods include selecting a subject for treatment (e.g., selecting a subject with insulin resistance). As noted herein, insulin resistance can be asymptomatic. Accordingly, direct diagnosis of insulin resistance can require assessing insulin sensitivity directly (e.g., by an insulin tolerance test (ITT), insulin sensitivity test (IST), and/or hyperinsulinemic-euglycemic clamp technique) and/or indirectly (e.g., by glucose uptake test (e.g., an oral glucose tolerance test (OGTT), a fasting glucose test, IV glucose tolerance test (FSIVGTT), and/or glucose clamp), e.g., as described in the Examples below (see also Muniyappa et al., Am. J. Physiol. Endocrinol. Metab., 294:E15-E26 (2008)).

In some embodiments, as subject can be defined insulin resistant if their insulin sensitivity index (SI) is: of the lowest 25% of a general population; of the lowest 10% of a non-obese, nondiabetic, normotensive Caucasian population; and/or of the lowest 10% of an obese, non-PCOS population. In some embodiments, a normal SI range can be calculated based on a sample of individuals who are not obese, have regular menstrual cycles, are not suffering from hirsutism, and have normal circulating androgen levels (see, e.g., Kanauchi et al., J. Clin. Endocrin. & Metab., 88:3444-3446 (2003).

In some embodiments, insulin sensitivity can be assessed based on results obtained using the oral glucose tolerance test (see Mari et al., Diabetes, 24:539-548 (2001)).

If blood glucose is assessed, blood glucose of 100-125 mg/dl demonstrates pre-diabetes or IFG. 126 mg/dl or above is consistent with the diagnosis of diabetes. In some embodiments, a diagnosis of insulin resistance in a subject can be made, and monitored, using any method known in the art, including the oral glucose tolerance test (OGTT), IV glucose tolerance test (FSIVGTT), insulin tolerance test (ITT), insulin sensitivity test (IST), and continuous infusion of glucose with model assessment (CIGMA), or the glucose clamp. See, e.g., Krentz, Insulin Resistance (Wiley-Blackwell, 2002); de Paula Martins et al., Eur. J. Obst. Gynecol. Reprod. Biol., 133 (2):203-207.

Methods for selecting a subject for treatment can also include selecting a subject with a risk factor for insulin resistance. Such risk factors can include, for example, being overweight, having a high waist circumference, having metabolic syndrome, obesity, pregnancy, infection or severe illness, stress, genetics, steroid use, and lack of exercise (sedentary life style).

Alternatively, or in addition, a subject can be selected based on the subject having a condition known to be associated with insulin resistance. Such conditions can include, for example, diabetes (e.g., type II diabetes), pre-diabetes, obesity, metabolic syndrome (syndrome X), cardiovascular disease (e.g., hypertension (e.g., up to 50% of patients with hypertension are estimated to have insulin resistance), arteriosclerosis (also known as atherosclerosis), skin lesions, reproductive abnormalities, polycystic ovary disease, hyperandrogenism, growth abnormalities, and fatty liver (hepatic steatosis). As stated above, the association between insulin resistance and such conditions allows clinicians to diagnose, or at least reliably assume, insulin resistance in a subject based on diagnosis of one or more of the conditions. Insulin resistance is also commonly assumed in subjects that are overweight, have a high waist circumference, and/or that are physically inactive.

In some embodiments, subject selection can include evaluating a subject for one or more of: weight, adipose tissue stores, adipose tissue morphology, insulin levels, insulin metabolism, glucose levels, thermogenic capacity, and cold sensitivity.

In some embodiments, obesity can refer to a disorder in a subject, wherein the subject's weight exceeds their ideal weight, according to standard tables, by 20% or more, e.g., 25%, 30%, 40%, and 50%, or more. Obese can also mean an individual with a body mass index (BMI) of about 30 or more, e.g., about 30-35 and/or about 35-40 or more. For example, a subject diagnosed with class I obesity has a BMI range of about 30-34.9. A subject with class II obesity has a BMI range of about 35.0-39.9. A subject with class III obesity has a BMI greater than about 40. Overweight can refer to a subject with a BMI range of about 25.0-29.9.

### Routes of Administration

One or more BMP agents (e.g., one or more BMP7 agents) can be administered to a subject by any means known in the art. For example, the BMP agents can administered according to any of the Food and Drug Administration approved methods, for example, as described in CDER Data Standards Manual, version number 004 (which is available at fda.give/cder/dsm/DRG/drg00301.htm). Exemplary systemic routes of administration can include, but are not limited to, parenteral, intravenous, intramuscular, intraperitoneal, intradermal, subcutaneous, percutaneous injection, oral, transdermal, and transmucosal administration. In some instances, the BMP agent can be administered to a site of adipose tissue, e.g., a subcutaneous or omentum adipose pad.

In some embodiments, BMP agents can be administered using a catheter and a pump. For example, BMP agents can be present in a refillable reservoir and can be propelled through the catheter by the pump. In some instances, the catheter can direct the BMP agents systemically, e.g., intravenously. Alternatively, the catheter can direct the BMP agents locally, e.g., to adipose tissue. The catheter and pump can be installed for long-term or short-term use.

Evaluating Insulin Resistance

In some embodiments, the methods can include evaluating insulin resistance in the subject before, during, and/or after the administration of one or more BMP agents (e.g., BMP7 agents). Such evaluations can be performed using any of the methods disclosed under the section titled “subject selection” above. For example, insulin resistance can be assessed directly (e.g., by an insulin tolerance test (ITT), insulin sensitivity test (IST), and/or hyperinsulinemic-euglycemic clamp technique) and/or indirectly (e.g., by glucose uptake test (e.g., an oral glucose tolerance test (OGTT), a fasting glucose test, IV glucose tolerance test (FSIVGTT), and/or glucose clamp). In some embodiments, insulin resistance can be assessed indirectly by measuring or quantifying a condition associated with insulin resistance. For example, if an insulin resistant subject is obese, a change (e.g., decrease) in the BMI of the subject can be assessed. In some instances, a decrease in the subject's BMI can indicate that an increase in insulin sensitivity has occurred. Similarly, a beneficial change in any of the insulin resistance associated conditions detailed in the subject selection section can be used as a marker of decreased insulin resistance. For example, a first level of a condition associated with insulin resistance prior to treatment, and a second level of the same condition after treatment, can indicate that a decrease in insulin resistance has occurred.

Evaluation can be performed at least 1 day, 2 days, 4, 7, 14, 21, 30 or more days before and/or after the administration. Evaluation can also be performed throughout treatment, e.g., while the subject is being administered one or more BMP agents (e.g., BMP7 agents).

Methods for Generating Brown Adipocytes from Insulin Resistant Cells and Cell Therapy Methods

In some embodiments, the present disclosure provides cell therapy methods. Such methods can include: obtaining an insulin resistant cell; contacting the insulin resistant cell with one or more BMP7 agents (e.g., one or more BMP7 agents) under conditions and for a time sufficient to cause the insulin resistant cell to differentiate (e.g., partially or completely differentiate) into a brown adipocyte. The methods can also optionally include administering (e.g., implanting) the resulting brown adipocyte into a subject (a subject suitable for treatment can be selected as disclosed in the section above entitled “subject selection”).

Insulin resistance interferes with adipogenesis, or the generation of fat cells (see, e.g., Yang et al., Biochem. Biophys. Res. Comm., 317:1045-1051, 2004; ClinicalTrials.gov identifier NCT00560469 at clinicaltrials.gov/ct2/show/NCT00560469 (last updated Feb. 17, 2009); Guilherme et al., Nature Reviews Molecular Cell Biology, 9:367-377, 2008). Insulin resistance also reportedly specifically impairs brown adipose tissue (BAT) adipogenesis (Yang et al., Obesity Res., 11:1182-1191, 2003). Specifically, the expression of genes associated with the generation of BAT (e.g., MASK, MAP3K5, PPARγ, pRb, RXRγ, and PGC-1) are decreased in insulin resistant subjects compared with insulin sensitive subjects. These observations suggest that a reduced brown adipocyte phenotype is associated with insulin resistance (Yang et al., Obesity Res., 11:1182-1191, 2003).

Both insulin and BMP signaling are important for adipocyte differentiation. It is reported that BMP-7 specifically promotes brown adipogenesis. As shown herein, analysis of gene expression profiles in cells in which the BMP-7 signaling pathway is activated (e.g., cells contacted with BMP-7) revealed cross-talk between BMP-7 and insulin signaling; specifically, a coordinated change in the expression of insulin signaling components upon induction of the BMP-7 signaling pathway. For example, upregulation of IRS-1, p85α subunit of P13-kinaseAkt, Grb2, Grb10, and down-regulation of the inhibitor of insulin signaling SOCS3 was observed in cells in which the BMP-7 signaling pathway is activated.

Accordingly, subjects with insulin resistance can suffer from a decreased ability to generate fat cells, or more specifically, brown adipose tissue (BAT), which is reported to be beneficial in obese patients and diabetics. This phenomenon is also problematic in vitro, for example, as it impedes the generation of BAT from insulin resistant cells. Such can be particularly problematic in cell therapy methods. For example, methods for obtaining cells from a diabetic or obese subject, which are differentiated to BAT, before being transplanted back into the subject are useful for the treatment of both conditions. Such methods are impeded, however, because cells derived from such subject are typically insulin resistant, and, therefore, cannot be efficiently differentiated to BAT.

In some embodiments, the disclose provides methods for generating brown adipocytes from insulin resistant cell in vitro. The resulting brown adipocytes can then be administered to a subject in need of additional brown adipocytes. In some embodiments, the resulting brown cells are administered to the subject from which the insulin resistant cells were obtained.

In some embodiments, the present disclosure provides methods for promoting brown adipogenesis in an insulin resistant cell. In some embodiments, these methods include obtaining an insulin resistant cell and enhancing BMP signaling (e.g., BMP-7 signaling) in the cell under conditions and for a time sufficient to increase the sensitivity of the cell to insulin. In some embodiments, BMP signaling (e.g., BMP-7 signaling) can be increased by 1%-10000% (e.g., 1%, 10%, 50%, 100%, 200% 300%, 500%, 1000%, 5000%, 10000% and more). In some embodiments, BMP signaling (e.g., BMP-7 signaling) can be increased for a period of from 1 hour to 168 hours and more (e.g., 1 hour, 6 hours, 12 hours, 24 hours, 48 hours, 72 hours, 96 hours, 120 hours, 168 hours and more). In some embodiments, BMP signaling (e.g., BMP-7 signaling) can be increased for 72 hours. In some embodiments, BMP signaling (e.g., BMP-7 signaling) can be increased indefinitely. In some embodiments, brown adipogenesis can be induced. In some embodiments, the cell can be transplanted into a subject as described below. Methods for detecting a BAT are described below.

Cell Types

The disclose includes the use of stem cells, pluripotent cells, and preadipocytes. Stem cells are cells pluripotent, e.g., are cells that are capable of both self-renewal and differentiation into many different cell lineages. Progenitor cells are a subset of stem cells with phenotypes similar to that of a stem cell. A progenitor cell is capable of self renewal and is typically multipotent. Preadipocytes are cells that are capable of differentiating into an adipocyte when subjected to the correct differentiation stimuli. In some embodiments, preadipocytes are cells that are capable of differentiating into an adipocyte when treated with one or more BMP agents (e.g., one or more BMP7 agents). In some embodiments, preadipocytes are cells that are capable of differentiating into a brown adipocyte when treated with one or more BMP agents (e.g., one or more BMP7 agents). Such preadipocytes that differentiate into brown adipocytes when treated with one or more BMP agents (e.g., one or more BMP7 agents) can be insulin resistant (e.g., prior to treatment with the one or more BMP7 agents).

In some embodiments, cells suitable for use in the methods described herein include any cell in which one or more of the cell's normal physiological responses to insulin are impaired or lost.

In some embodiments, such cells include cells that would otherwise be capable of differentiating into an adipocyte, but that have an impaired ability to differentiate into an adipocyte (e.g., a white or brown adipocyte). In some embodiments, such cells include cells that would otherwise be capable of differentiating into an adipocyte, but that cannot differentiate into an adipocyte (e.g., a white or brown adipocyte). In some embodiments, such cells include insulin resistant adipocytes (e.g., brown and/or white adipocytes). In some embodiments, such cells include brown preadipocytes. In some embodiments, such cells include white preadipocytes. Examples of such cells include, but are not limited to, insulin resistant stem cells and progenitor cells isolated from a variety of tissues and organs including, but not limited to, for example, skeletal muscle, prostate, dermis, the cardiovascular system, mammary gland, liver, neonatal skin, calvaria, bone marrow, the intestine, and adipose tissue (e.g., adipose tissue deposits), e.g., isolated from an insulin resistant subject.

In some embodiments, a preadipocyte can be identified based upon the detection of one or more of the following markers: PPARγ, C/EBPα, C/EBPβ, C/EBPδ, Glut4, aP2, FAS, and/or adiponectin. In some embodiments, a brown adipocyte can be identified based upon the detection of one or more of the following brown adipocyte specific markers: UCP1, PRDM16, PGC-1a, PGC-1b, ERRa, Tfam, ATPase f1a1, and/or COX7a1. Any one or more of these markers can be assessed, e.g., by detecting the protein or mRNA of the marker.

The term “primary cell” includes cells present in a suspension of cells isolated from a mammalian tissue source (prior to their being plated, i.e., attached to a tissue culture substrate such as a dish or flask), cells present in an explant derived from tissue, both of the previous types of cells plated for the first time, and cell suspensions derived from these plated cells. The term “secondary cell” or “cell strain” refers to cells at all subsequent steps in culturing. Secondary cells are cell strains that consist of secondary cells that have been passaged one or more times.

Primary and secondary cells can be obtained from a variety of tissues and include cell types which can be maintained and propagated in culture. Primary cells are preferably obtained from the individual or animal to whom the BMP-treated cells are administered. However, primary cells can also be obtained from a donor (e.g., an individual other than the recipient, typically of the same species, preferably an immunologically compatible individual). Methods for obtaining and culturing such cells are known in the art.

The cells can be autologous, allogeneic, or xenogeneic. In some embodiments, methods described herein can include obtaining a population of cells from a subject, optionally culturing and/or enriching the cells to obtain a purified population of cells, treating the cells with an agent that enhances BMP signaling as described herein to activate the cells, and implanting the cells in the same subject from which they were removed.

In some embodiments, the cells are allogeneic or xenogeneic; if necessary, immune suppression, as known in the art, can be administered to prevent rejection of the cells.

In some embodiments, the present disclosure provides methods for increasing insulin sensitivity in a cell. In some embodiments, these methods include obtaining an insulin resistant cell and enhancing BMP signaling (e.g., BMP-7 signaling) in the cell under conditions and for a time sufficient to increase the sensitivity of the cell to insulin. In some embodiments, BMP signaling (e.g., BMP-7 signaling) can be increased by 1%-10000% (e.g., 1%, 10%, 50%, 100%, 200%, 300%, 500%, 1000%, 5000%, 10000% and more). In some embodiments, BMP signaling (e.g., BMP-7 signaling) can be increased for a period of from 1 hour to 168 hours and more (e.g., 1 hour, 6 hours, 12 hours, 24 hours, 48 hours, 72 hours, 96 hours, 120 hours, 168 hours and more). In some embodiments, BMP signaling (e.g., BMP-7 signaling) can be increased for 72 hours. In some embodiments, BMP signaling (e.g., BMP-7 signaling) can be increased indefinitely.

In some embodiments, an alteration (e.g., increase) in insulin sensitivity can be assessed following enhancement of BMP signaling (e.g., BMP-7 signaling) in the cell. For example, insulin sensitivity can be assessed by measuring the levels of one or more of the genes listed in Table 1 prior to treatment, during treatment, and/or post treatment, where an increase in the level of the gene (e.g., IRS-1) indicates that the insulin sensitivity of the cell has increased.

In some embodiments, the insulin resistant cell is a substantially pure population of insulin resistant cells, wherein at least 50% (e.g., 50%, 60%, 70%, 80%, 90%, 95%, 99%, or more) cells are insulin resistant.

In some embodiments, the insulin resistant cell is in a subject, and the methods include identifying a subject who has insulin resistance, administering a compound that enhances BMP signaling (e.g., BMP-7 signaling), and optionally evaluating the effect of the treatment on insulin resistance.

Cell Therapy

In certain embodiments, methods described herein can include implanting one or more (e.g., a population) of the BMP treated cells described herein into a subject selected as disclosed above. The BMP-treated cells can be implanted directly or can be administered in a scaffold, matrix, or other implantable device to which the cells can attach (examples include carriers made of, e.g., collagen, fibronectin, elastin, cellulose acetate, cellulose nitrate, polysaccharide, fibrin, gelatin, and combinations thereof). In general, the methods include implanting a population of BMP-treated cells comprising a sufficient number of cells to promote brown adipogenesis in the subject, e.g., to increase the amount of BAT in the subject by at least 1%, e.g., 2%, 5%, 7%, 10%, 15%, 20%, 25% or more.

In some embodiments, the methods include providing a purified population of insulin resistant cells, (e.g., a population of cells in which at least 60%, e.g., 70%, 80%, 90% or more of the cells are insulin resistant); and contacting these cells with a BMP agent (e.g., a BMP7 agent), thereby generating BMP-treated cells.

Methods described herein can include implanting a population of BMP-treated cells, e.g., as described herein, into a subject to be treated, wherein said population of BMP-treated stem cells, or their progeny (i.e., daughter cells), undergo brown adipogenesis. Once implanted, the cells will generally undergo adipogenesis, generating BAT in the subject.

These cell therapy methods are useful, e.g., for the treatment of obesity and insulin resistance in a subject, or for treating a disease associated with a lack of mitochondria, e.g., diabetes, cancer, neurodegeneration, and aging.

Methods for implanting the populations cells that have been treated using the methods described herein are known in the art, e.g., using a delivery system configured to allow the introduction of cells into a subject. In general, the delivery system can include a reservoir containing a population of cells, and a needle in fluid communication with the reservoir. Typically, the population of cells will be in a pharmaceutically acceptable carrier, with or without a scaffold, matrix, or other implantable device to which the cells can attach (examples include carriers made of, e.g., collagen, fibronectin, elastin, cellulose acetate, cellulose nitrate, polysaccharide, fibrin, gelatin, and combinations thereof). Such delivery systems are also within the scope of the invention. Generally, such delivery systems are maintained in a sterile manner. Various routes of administration and various sites (e.g., renal sub capsular, subcutaneous, central nervous system (including intrathecal), intravascular, intrahepatic, intrasplanchnic, intraperitoneal (including intraomental), intramuscularly implantation) can be used. Generally, the cells will be implanted into the subject subcutaneously. In some embodiments, the population of cells that is implanted includes at least 10⁷, 10⁸, 10⁹, or more cells.

Where non immunologically compatible cells are used, an immunosuppressive compound e.g., a drug or antibody, can be administered to the recipient subject at a dosage sufficient to achieve inhibition of rejection of the cells. Dosage ranges for immunosuppressive drugs are known in the art. See, e.g., Freed et al., N. Engl. J. Med. 327:1549 (1992); Spencer et al., N. Engl. J. Med. 327:1541 (1992); Widner et al., N. Engl. J. Med. 327:1556 (1992)). Dosage values may vary according to factors such as the disease state, age, sex, and weight of the individual.

In some embodiments, the methods include contacting, administering or expressing one or more other compounds in addition to the BMP-treated cells, e.g., peroxisome proliferator-activated receptor gamma (PPARγ), Retinoid X receptor, alpha (RXRα), insulin, T3, a thiazolidinedione (TZD), retinoic acid, another BMP protein (e.g., BMP-1 or BMP-3), vitamin A, retinoic acid, insulin, glucocorticoid or agonist thereof, Wingless-type (Wnt), e.g., Wnt-1, Insulin-like Growth Factor-1 (IGF-1), or other growth factor, e.g., Epidermal growth factor (EGF), Fibroblast growth factor (FGF), Transforming growth factor (TGF)-α, TGF-13, Tumor necrosis factor alpha (TNFα), Macrophage colony stimulating factor (MCSF), Vascular endothelial growth factor (VEGF) and/or Platelet-derived growth factor (PDGF). In other embodiments, the compound can be a BMP-2, -4, -5, -6, and/or -7 protein as described herein or a portion thereof linked with a heterologous polypeptide sequence, e.g., a second BMP protein, to form a chimeric molecule or fusion protein. In some embodiments, the methods include administering the compound in combination with a second treatment, e.g., a second treatment for obesity or a related disorder such as diabetes. For example, the second treatment can be insulin, orlistat, phendimetrazine, and/or phentermine.

### EXAMPLES

The invention is further described in the following examples, which do not limit the scope of the invention described in the claims.

The following data support, inter alia, that BMP-7 treatment can promote insulin sensitivity and rescue an insulin resistant cell's ability to differentiate into a brown adipocyte.

### Example 1

### BMP7 Alters the Expression of Insulin Signaling Components

Both insulin and BMP signaling pathways are essential for brown adipogenesis. The following experiments were performed to determined whether a crosstalk between these two pathways is involved in driving the brown adipogenic program.

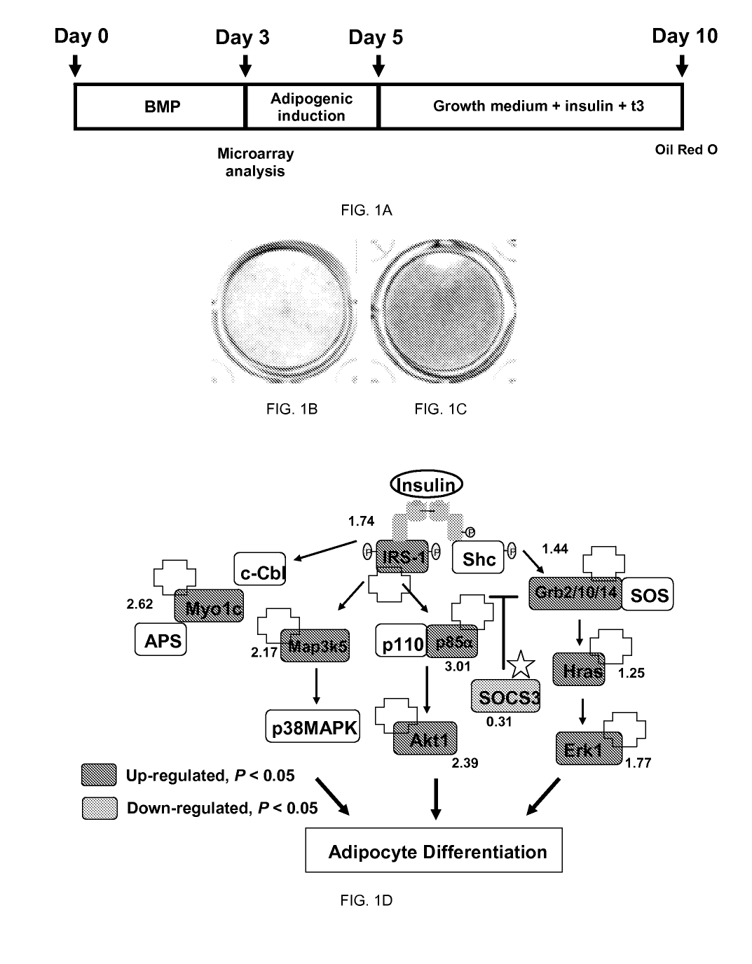

C3H10T1/2 cells were cultured in DMEM supplemented with 10% FBS. At 60% confluency, the cells were treated with 3.3 nM BMP7 or vehicle for 3 days, and then subjected to standard adipogenic differentiation. The cells were stained by Oil Red O on day 10 (FIGS. 1A-1B), and mRNAs were isolated on day 3 and subjected to microarray analysis using Affymetrix M430A 2.0 chips.

Microarray was performed as follows. Briefly, RNA samples from four independent experiments were analyzed by microarray using the Affymetrix GeneChip mouse 430A. A total of 8 chips were used. Intensity values were quantitated by MAS 5.0 software (Affymetrix). All chips were subjected to global scaling to a target intensity of 1, 500 to take into account the inherent differences between the chips and their hybridization efficiencies. Significance of gene expression was determined by Student's t-test using the p value less than or equal to 0.05 as the threshold.

The results are shown in Table 1 and are illustrated in FIG. 1C (Numbers on the side of the gene names indicate the fold change of a given gene in BMP7-treated cells relative to control, which correspond to the most significant p value among different probe sets). P values for statistically significant increases or decreases are shown in bold.

TABLE 1
Gene Expression Profiles in BMP-7 Treated Cells

| | | | Incr/ | P |
|---|---|---|---|---|
| Gene Name | Gene Symbol | BMP7/Control | Decre | Value |
| insulin receptor substrate 1 | Irs1 | 1.74 | Incr | 0.00264 |
| phosphatidylinositol 3-kinase, | Pik3r1/p85 alpha | 3.01 | Incr | 0.00847 |
| regulatory subunit, polypeptide 1 | | | | |
| phosphatidylinositol 3-kinase, | Pik3r1/p85 alpha | 1.97 | Incr | 0.02279 |
| regulatory subunit, polypeptide 1 | | | | |
| phosphatidylinositol 3-kinase, C2 | Pik3c2a | 2.74 | Incr | 0.01979 |
| domain containing, alpha | | | | |
| polypeptide | | | | |
| phosphatidylinositol 3-kinase, C2 | Pik3c2a | 1.30 | Incr | 0.21098 |
| domain containing, alpha | | | | |
| polypeptide | | | | |
| thymoma viral proto-oncogene 1 | Akt1 | 2.39 | Incr | 0.02830 |
| thymoma viral proto-oncogene 1 | Akt1 | 1.29 | Incr | 0.07117 |
| thymoma viral proto-oncogene 2 | Akt2 | 2.20 | Incr | 0.06887 |
| thymoma viral proto-oncogene 2 | Akt2 | 1.26 | Incr | 0.18253 |
| serum/glucocorticoid regulated | Sgk | 1.77 | Incr | 0.04571 |
| kinase | | | | |
| growth factor receptor bound | Grb2 | 1.44 | Incr | 0.04582 |
| protein 2 | | | | |
| growth factor receptor bound | Grb2 | 1.18 | Incr | 0.24853 |
| protein 2 | | | | |
| growth factor receptor bound | Grb10 | 2.81 | Incr | 0.01284 |
| protein 10 | | | | |
| growth factor receptor bound | Grb10 | 0.94 | Incr | 0.66449 |
| protein 10 | | | | |
| growth factor receptor bound | Grb10 | 1.85 | Incr | 0.00507 |
| protein 10 | | | | |
| growth factor receptor bound | Grb14 | 2.08 | Incr | 0.02243 |
| protein 14 | | | | |
| Harvey rat sarcoma virus | Hras1 | 1.25 | Incr | 0.04598 |
| oncogene 1 | | | | |
| mitogen activated protein kinase 3 | Mapk3/Erk1/p44 | 1.77 | Incr | 0.00755 |
| | MAPK | | | |
| mitogen activated protein kinase | Map3k5/ASK1 | 2.17 | Incr | 0.04706 |
| kinase kinase 5/apoptosis signal- | | | | |
| regulating kinase | | | | |
| adaptor protein with pleckstrin | Aps | 2.70 | Incr | 0.01483 |
| homology and src | | | | |
| adaptor protein with pleckstrin | Aps | 1.39 | Incr | 0.37964 |
| homology and src | | | | |
| myosin IC | Myo1c | 2.62 | Incr | 0.03341 |
| suppressor of cytokine signaling 3 | Socs3 | 0.38 | Decr | 0.00550 |
| suppressor of cytokine signaling 3 | Socs3 | 0.31 | Decr | 0.00876 |
| suppressor of cytokine signaling 3 | Socs3 | 0.43 | Decr | 0.01882 |

It is reported that BMP7 triggers commitment of the multipotent mesenchymal C3H10T1/2 cells to a brown adipocyte lineage and transplantation of BMP7-treated C3H10T1/2 cells into athymic nude mice results in formation of brown fat tissue in vivo.

As shown in FIG. 1C and Table 1, treatment of C3H10T1/2 cells with BMP7 for three days followed by standard adipogenic cocktail led to a marked increase in lipid accumulation (FIG. 1B).

As shown in FIG. 1C and Table 1, microarray revealed that BMP7 significantly regulated several key components of the insulin signaling pathway in these mesenchymal cells. These include up-regulation of IRS-1, p85 subunit of phosphotidylinositol 3 kinase (PI3-kinase), thymoma viral proto-oncogene (Akt), growth factor receptor-bound protein 2 (Grb2), growth factor receptor-bound protein 10 (Grb10), and extracellular signal-regulated kinase 1 (Erk1), and down-regulation of suppressor of cytokine signaling 3 (SOCS3), an inhibitor of insulin signaling. It has been shown that activation of the PI3K-Akt pathway is required for insulin-induced adipogenesis (1, 34, 57, 59), while adipocyte differentiation is associated with downregulation of SOCS3 expression (11). Thus, the coordinated regulation of insulin signaling components by BMP7 suggests that BMP7-induced adipogenesis is mediated at least partly through enhancing insulin signaling.

As shown in Table 1 and FIG. 1C, the expression levels of a number of genes were increased following treatment. Among these gene were IRS-1, p85α subunit of P13-kinase, Akt, Grb2, Grb10, and Erk1.

These observations support that BMP7 promotes a coordinated change in insulin signaling components in fibroblasts and supports the existence of cross-talk between insulin and BMP signaling systems in brown adipogenesis.

### Example 2

### BMP7 Treatment Rescued Brown Adipocyte Differentiation in IRS-1KO Cells

To further investigate the cross-talk between insulin and BMP signaling systems in brown adipogenesis, we examined the effect of BMP7 in insulin receptor substrate-1 (IRS-1) deficient brown preadipocytes, which exhibit a severe defect in differentiation.

Establishment of WT and IRS-1KO brown preadipocytes isolated from new born wild type and IRS-1KO mice and their differentiation into brown adipocytes were performed as previously described (Fasshauer et al., Mol. Cell. Biol., 175:727-733 (2002); Tseng et al., Nature, 454:1000-1004 (2008); Tseng et al., Mol. Cell. Biol., 24:1918-1929 (2004); Tseng et al., J. Biol. Chem., 277:31601-31611 (2002)), except that the cells were pre-treated with 3.3 nM BMP7 or vehicle for 3 days before induction of differentiation as indicated. Briefly, brown preadipocytes were cultured in DMEM supplemented with 10% FBS (Gemini Bio-Products). Adipocyte differentiation was induced by incubating 100% confluent cells in DMEM supplemented with 10% FBS, 0.5 uM Dexmethasone, 0.5 mM IBMX, 20 nM insulin, 0.125 mM indomethacin, 1 nM T3 for 2 days, and then switched to DMEM+10% FBS supplemented with 20 nM insulin and 1 nM T3 and renewed every second day until the cells were harvested at indicated time points. As a control, the wild-type (WT) brown preadipocytes were also treated with vehicle, then induced to undergo adipocyte differentiation

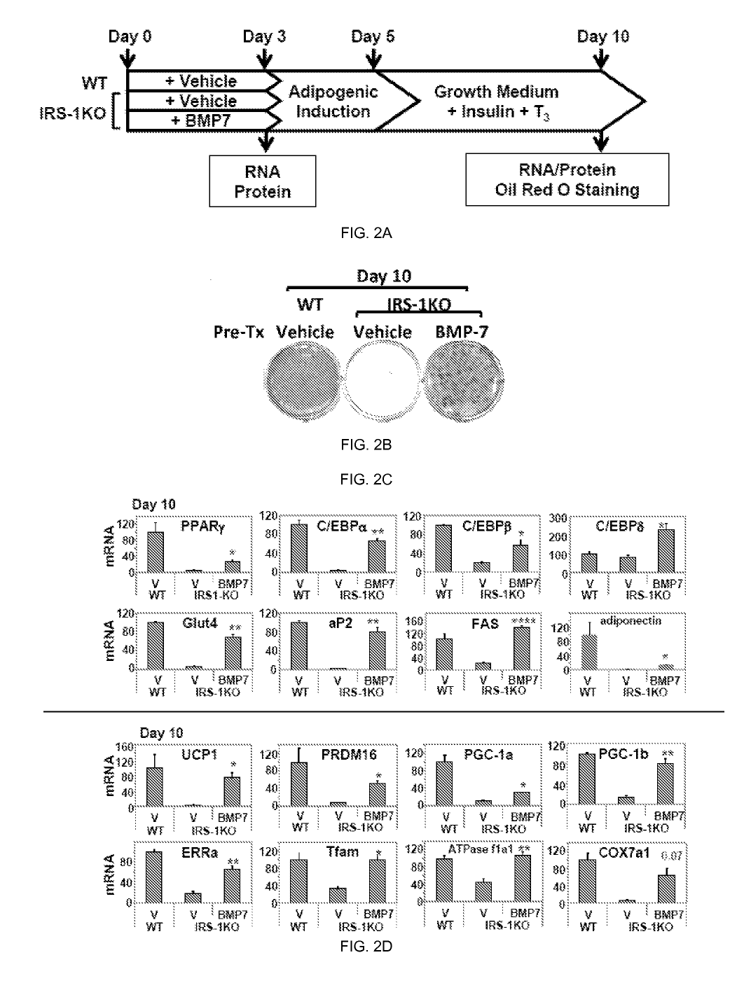

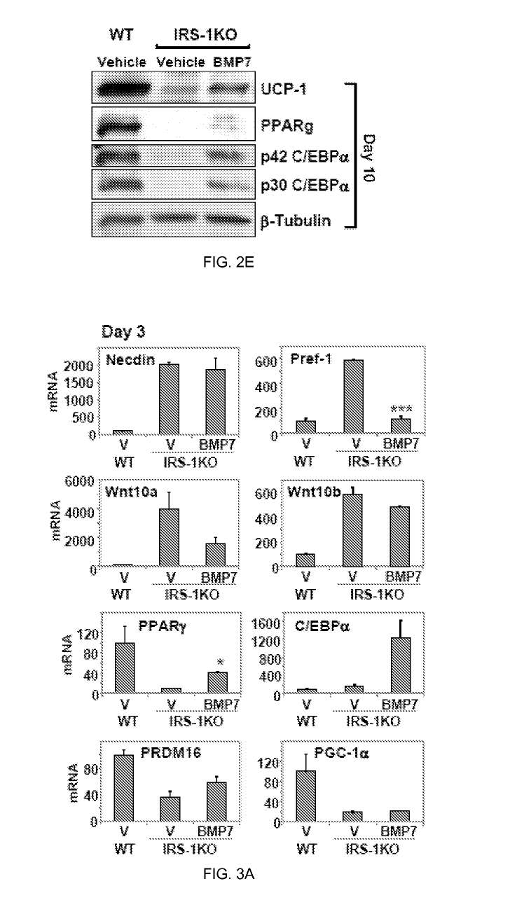

Experiments were performed as illustrated in FIG. 2A. Briefly, isolated wild type or IRS1-KO were pre-treated with vehicle or BMP7 for 3 days. Adipogenesis was then induced using the adipgogenesis inducing media MX/Dex/Insulin/T3/Indomethacin (Tseng et al., Mol. Cell. Biol., 24:1928-1929, 2004) as shown in FIG. 2A. On day 10, dishes were stained with Oil Red O (which stains lipid red).

As shown in FIG. 2B, BMP7 treatment effectively restored the differentiation defect in IRS-1KO cells compared to that of wild type cells achieved by a standard differentiation protocol and as judged by Oil Red O staining. IRS-1 knock out cells not pretreated with BMP-7 did not stain for Oil Red O suggesting that these cells did not undergo adipogenesis.

Gene expression levels in BMP-7 treated IRS-KO cells was analyzed using quantitative RT-PCR. RNA was then isolated on Day 3 and Day 10 and analyzed by Q-RT-PCR. As shown in FIG. 2C, Q-RT-PCR analysis revealed that BMP7 treatment completely or partially restored the expression of key adipogenic transcription factors PPARγ, C/EBPα, C/EBPβ, C/EBPδ, as well as markers for mature adipocytes, including aP2, Glut4, FAS and adiponectin in IRS-1KO cells at day 10 of this program.

As shown in FIG. 2D, BMP7 treatment in IRS-1KO cells and adipogenic induction led to near complete restoration of UCP1 expression at day 10 (FIG. 2D). This is accompanied by a significant increase in the expression of several key regulators of brown adipogenesis, including PR domain containing 16 (PRDM16), and nuclear co-activators peroxisome proliferative activated receptor gamma coactivator 1α (PGC-1α), PGC-1β, and the orphan nuclear receptor estrogen-related receptor a (ERRα). The transcriptional complex consisting of ERRα and PGC-1α has been shown to play an essential role in the regulation of mitochondrial gene expression and biogenesis (Mootha et al., Proc. Natl. Acad. Sci., 101:6570-6575 (2004); Rangwala et al., Biochem. Biophys. Res. Com., 357:7230-7242 (2003); Schreiber et al., Proc. Natl. Acad. Sci., 101:6472-6477 (2004)). Thus, expression of genes involved in mitochondrial replication and oxidative phosphorylation was increased in cells treated with BMP7, including mitochondrial transcription factor A (Tfam), cytochrome c oxidase, subunit VIIa 1 (COX7a1), and ATP synthase, H+ transporting, mitochondrial F1 complex, alpha subunit, isoform 1 (ATPase f1a1) (see FIG. 2D).

Results similar to those reported in FIGS. 2C and 2D were observed at the 3 day time point.

Expression of certain markers was also confirmed by Western blotting. Briefly, cultured cells were harvested in protein sample buffer. 50 μg total proteins were loaded in each lane in SDS-PAGE for western blotting analysis. Samples were then probed with the following primary antibodies used in western blotting were: UCP1 (Santa Cruz sc-6528), PPARγ (Santa Cruz sc-7273), C/EBPα (Santa Cruz sc-61), and β-Tubulin (Cell Signaling Cat 2146). Medium was harvested from cultures of WT and IRS-1KO overexpressing BMP7 and analyzed for BMP7 expression level by ELISA using Human BMP7 DuoSet ELISA Development kit from R&D Systems following the manufacturer-provided protocol.

As shown in FIG. 2E, Western blotting analysis further confirmed that BMP7 pre-treatment restored protein expression of UCP1, PPARλ, and C/EBPα.

These data suggest that BMP7 can reverse the differentiation defect in brown preadipocytes with impairment in insulin signaling.

To further explore the molecular mechanisms underlying these processes, mRNA and protein expression was analyzed in IRS-1KO cells treated with BMP7 or vehicle for three days. It is reported that prior to entering the adipogenic program, preadipocytes need to be released from suppression controlled by a number of adipogenic inhibitors such as necdin (Goldfine et al., Diabetes, 55:640-650 (2006)), Wnt family proteins (Ross et al., Science, 289:950-953 (2000)), and Pref-1 (Sul, Mol. Endocrinol., 23:1717-1725 (2009)). Disruption of insulin signaling by knocking down IRS-1 led to increased expression of necdin, Wnt and Pref-1, and resulted in a blockage of the transcriptional cascade necessary for adipogenesis (Tseng et al., Nat. Cell. Biol., 7:601-611 (2005)).

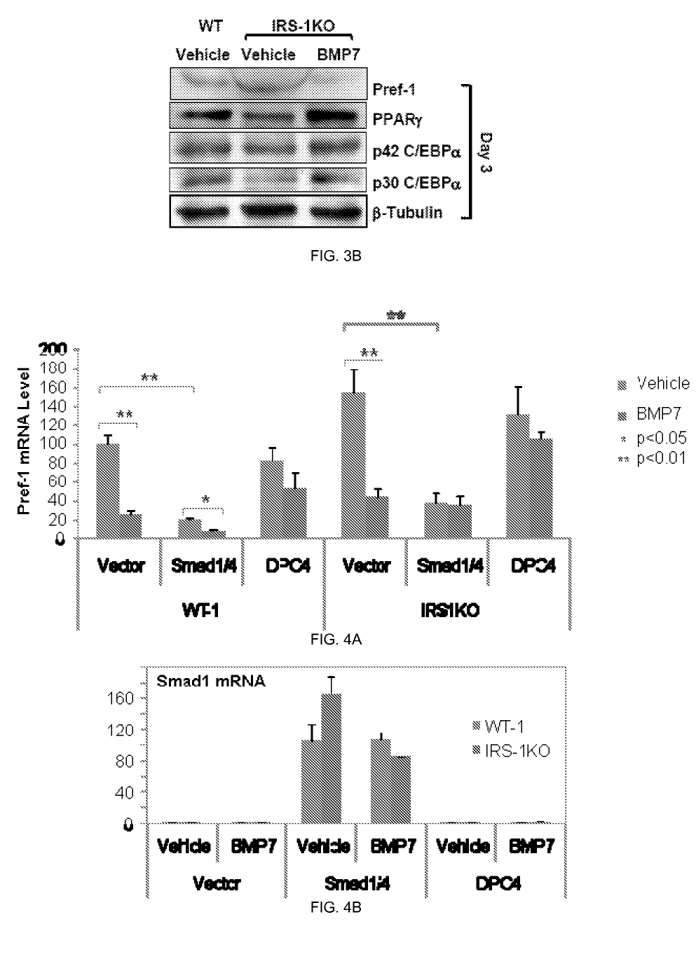

As shown in FIGS. 3A-3B, treatment of IRS-1KO cells with BMP7 for 3 days did not affect the expression of necdin and Wnt10b, but tended to reduce the expression of Wnt10a, although it did not reach statistical significance. Importantly, BMP7 completely reversed the elevated expression of Pref-1 in IRS-1KOcells at both mRNA and protein levels.

In addition to removal of suppression, three days of BMP7 pre-treatment also significantly induced mRNA and protein expression for two key adipogenic transcription factors PPARλ and C/EBPα in IRS-1KO cells (see FIGS. 3A-3B). Expression of the early brown adipogenic regulator PRDM16 was reduced by 50% in IRS-1KO cells, and this was partially rescued by BMP7 treatment (FIG. 3A). The reduced levels of PGC-1α in IRS-1KO cells was not altered at this early time point, but it was partially restored in BMP7-treated cells at the later time point (FIG. 2D).

These results support that the rescue of brown adipocyte differentiation by BMP7 in IRS-1KO cells is mediated at least in part by suppressing Pref-1 expression.

The data presented herein support that BMP-7 can bypass upstream inhibitors of brown adipogenesis and directly inhibit pref-1 expression and thereby initiate the brown adipogenic program.

### Example 3

### Role of Smad in BMP-7 Induced Insulin Sensitivity

BMP-7 regulates gene transcription via activation of Smad proteins (Chen et al., Growth Factors 22:233-241, 2004). The association between BMP-7, Smad, insulin resistance, and brown adipogenesis was investigated in mouse embryonic fibroblast cells from wild type or IRS1-KO animals.

Briefly, wild type and IRS1-KO mouse brown preadipocytes were transfected with pCS2-Flag-Smad1 and pCS2-Flag-Smad4, or pCS2-DPC4 (dominant negative form of Smad4). Six hours post transfection, cells were treated with vehicle or BMP7 for 72 hours. RNA was then isolated and analyzed for Pref-1, Smad1, Smad4 expression by qPCR.

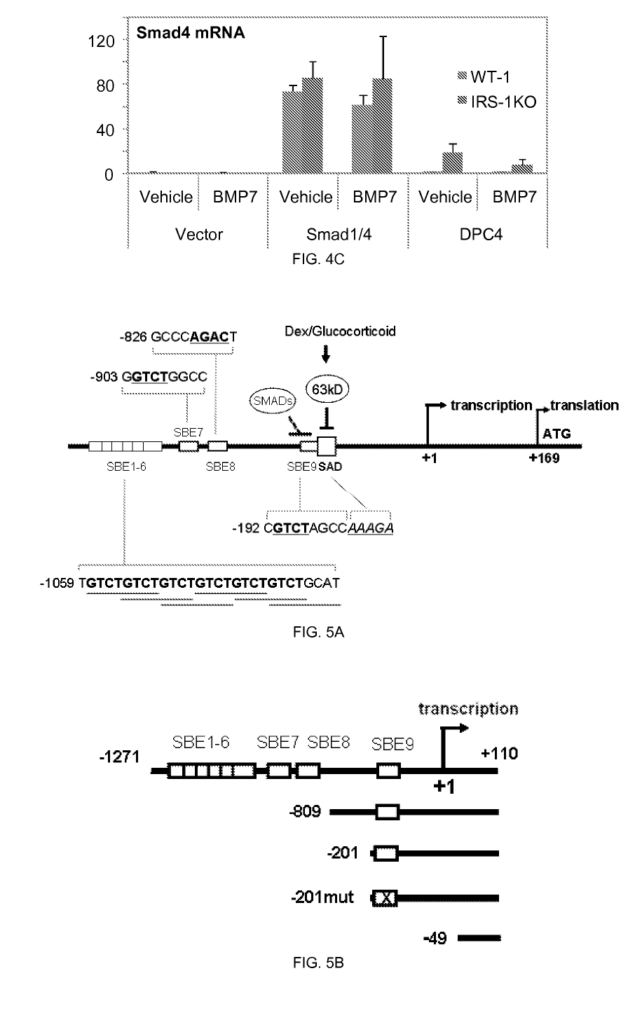

As shown in FIG. 4B-C, Pref-1 mRNA expression was decreased in BMP-7 treated wild type and IRS1-KO cells overexpressing all Smad constructs. Increased Smad1 and Sma4 mRNA expression levels were also observed in IRS1-KO vehicle treated Smad1/4 overexpressing cells compared to vehicle treated cells. Increased Smad4 mRNA expression was also observed in BMP7 treated IRS1-KO Smad1/4 overexpressing cells compared to vehicle treated cells. Decreased Smad1 expression was observed in BMP7 treated IRS1-KO Smad1/4 overexpressing cells compared to vehicle treated cells. These data suggest that BMP-7 utilizes the Smad pathway to suppress expression of Pref-1 mRNA

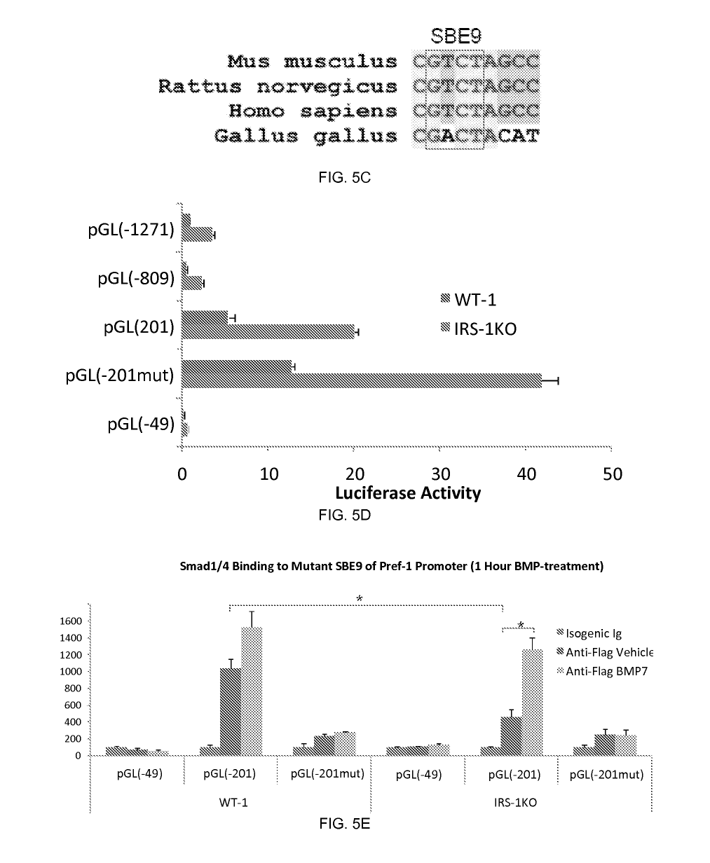

To characterize the region of the Pref-1 promoter that is important in communicating the BMP-7 signal, deletion analysis of the Pref-1 promoter (see FIG. 5A) was performed according to the strategy shown in FIG. 5B. Briefly, sequential deletion mutants of the 1.3 kb mouse Pref-1 promoter were cloned in pGL4.1basic vector. Wild type and IRS1-KO cells were then transfected with these pGL-pref promoter constructs. Six hours post transfection, cells were treated with BMP7 for 24 hours and then lyzed for luciferase assay. As shown in FIG. 5B, the Pref-1 promoter was most active in IRS1-KO cells from the −201 mutant.

Binding of Smad1/4 to the Pref-1 promoter was also analyzed. Briefly, wild type and IRS1-KO cells were co-transfected with pGL(−201) or pGL(−201mut) along with pCS2-Flag-Smad1/pCS2-Flag-Smad4. Eighteen hours after transfection, cells were treated with vehicle or BMP7 for 1 hour, followed by 10-minutes 1% formaldehyde. The cells were lysed, chopped by sonication for ChIP assay. Anti-flag antibody and Isogenic Immunoglobulin (as mock control) were used for ChIP. Precipitated DNA were analyzed by qPCR using primers flanking SBE9.10% of ChIP input sample were also quantified by qPCR for normalization. The data presented in FIGS. 4C-D confirms a direct binding of Smad1 and Smad4 to the proximal region of Pref-1 promoter, thereby regulating its gene expression.

These results support that the promoter proximal SBE (−192/184) is critical in mediating BMP-7's suppressive effect on Pref-1.

### Example 4

### BMP-7 Treated Cells Undergo Spontaneous Brown Adipogenesis

The observation that BMP pre-treatment specifically downregulated Pref-1 expression in IRS-1KO cells prompted analysis of whether BMP signaling may directly target the pref-1 promoter to repress its expression at the early stage of differentiation.

To determine whether inhibition of Pref-1 expression by BMP7 is mediated by the Smad signaling pathway, WT and IRS-1KO cells were transfected with Smad1 and Smad4 expression vectors, a vector expressing dominant negative form of Smad4 (DPC4) (22), or the empty vector as control.

Transfections and luciferase assays were done using Lipofectamine 2000 (Invitrogen) and Dual-luciferase system (Promega), respectively, following the protocols recommended by the manufacturers. Briefly, WT and IRS-1KO cells were transfected with mouse pref-1 promoter constructs pGL(−201), pGL(−201mut) and pGL(−49). Six hours after transfection, cells were treated with BMP7 for 24 hours and then assayed for luciferase activity.

As shown in FIGS. 4B-C, transfection of the Smad constructs resulted in a 70-160 fold increase in Smad1 and Smad4 expression over the basal levels. After culturing such transfected cells in the presence or absence of BMP7 for 3 days, mRNA was isolated and Pref-1 gene expression analyzed.

As shown in FIG. 4A, consistent with the findings described above, BMP7 treatment resulted in a near 80% reduction in Pref-1 expression in both WT and IRS-1KO cells transfected with empty vector alone. Overexpression of Smad1/4 in these cells significantly suppressed Pref-1 expression at basal state, and BMP7 treatment further decreased Pref-1 expression in WT cells, but not IRS-1KO cells. Importantly, when the dominant negative form of Smad4, DPC4, was expressed in either WT or IRS-1KO cells, the inhibitory effect of BMP7 on Pref-1 expression was blunted, indicating that BMP7 worked through the Smad1/4 complex to inhibit Pref-1 expression.

### Example 5

### Smad1/4 Binds to the Smad Binding Element of pref-1 Promoter and Inhibits its Expression

Experiments were performed to determine whether the Smad1/4 complex directly suppress pref-1 promoter activity.

Bioinformatics analysis revealed the presence of 9 putative Smad Binding Elements (SBEs, herein referred as SBE1-SBE9) within the 1.3 kb pref-1 promoter region (from −1271 to +110). The first 6 SBEs consisting of the repeated core sequence 5′-GTCT-3′ overlapped each other, while SBE8 was in the reversed direction. Interestingly, the promoter-proximal SBE, SBE9, was located right next to the SAD (Suppression in Adipocyte Differentiation) element which was previously reported to bind a 63 KD protein (p63) in response to dexamethasone treatment to repress pref-1 promoter activity (see FIG. 5A). Alignment of the putative SBE sequences in the pref-1 promoter from different species revealed that the SBE9 sequences shared 100% identity among mice, rats and humans (i.e., GTCT), while the other SBEs exhibited a different degree of variation (see FIG. 5C).

To determine whether these putative SBEs are required for regulation of Pref-1 expression, luciferase reporter constructs were generated harboring sequential deletions of the 1.3 kb pref-1 promoter and a point mutation in SBE9 (see FIG. 5B). These constructs were transfected into both WT and IRS-1KO cells. As shown in FIG. 5D, deletion of SBE1 to SBE8 slightly decreased reporter gene activity in both WT and IRS-1KO cells. This observation suggests that SBE1 to SBE8 do not play a role in suppression of Pref-1 expression by the Smad pathway. However, introducing a mutation of two bases in the SBE9 significantly increased the promoter activity by two-folds in both WT and IRS-1KO cells (see FIG. 5D). This observation suggests that SBE9 functions as an inhibitory element for Pref-1 expression. Further deletion of the promoter sequences from −201 to −49 completely abolished luciferase activity (FIG. 5D), indicating this region contains key regulatory elements essential for Pref-1 gene expression.

Determination of whether Smad1/4 can directly bind to SBE9 was performed using modified ChIP analysis. Briefly, WT and IRS-1KO cells were co-transfected with pGL(−201), pGL(−201mut) and pGL(−49) constructs along with pCS2-Flag-Smad1/pCS2-Flag-Smad4. Eighteen hours after transfection, cells were treated with vehicle or BMP7 for 1 hour. Cells were then incubated in 1% formaldehyde at room temperature for 10 minutes for crosslinking, followed by addition of 1 M Glycine to a final concentration of 0.125M, and incubated at room temperature for 10 minutes. The cells were washed twice with cold PBS, and harvested in ChIP lysis buffer (0.1% SDS, 1% Triton X-100, 0.15 M NaCl, 1 mM EDTA and 20 mM Tris, pH 8). The samples were then subjected to sonication for 8 minutes to ensure that DNA was fragmented to the size range 100 bp to 1000 bp. 100 μg sample, 20 μg Protein G (Amersham) and 1 μg anti-flag antibody (Sigma) or isogenic immunoglobulin (as mock control) were used for each ChIP reaction to precipitate Smad1/4 protein. Co-precipitated DNA were subjected to a series of washes in buffers of different salt concentrations and then de-crosslinked in buffer (1% SDS, 0.1M NaHCO3, 0.2M NaCl) at 65° C. for 4 hours. After phenol chloroform extraction of proteins, the DNA was precipitated and dissolved in nuclease-free water, and then analyzed by Q-RT-PCT using primers specific for the transgene SBE9 that can distinguish SBE9 sequence of the transfected pref-1 promoter construct from that of the endogenous pref-1 promoter. 10% of the ChIP input samples (without precipitation and washing) were also subjected to the same procedures and quantified by Q-RT-PCT as an internal control for normalization.

WT and IRS-1KO cells were co-transfected with flag-tagged Smad1 and Smad4 expression vectors along with pref-1 promoter constructs pGL(−49), pGL(−201) (containing the wild type SBE9) and pGL(−201mut) (containing the mutated SBE9). The cells were treated with BMP7 or vehicle, and then subjected to ChIP analysis using the anti-flag antibody to pull down protein-DNA complex. As shown in FIG. 5E, the pref-1 promoter construct containing SBE9 appeared to bind Smad1/4 complex as quantified by Q-RT-PCT using specific primers flanking SBE9. Binding of the Smad1/4 complex to SBE9 (pGL(−201)) was increased by 1.5- and 2.7-fold by BMP7 treatment in WT and IRS-1KO cells, respectively. Importantly, this binding was almost completely abolished when SBE9 was mutated. Notably, at the basal state (vehicle treatment), the binding of Smad1/4 complex to SBE9 in IRS-1KO cells was about half of that in WT cells, indicating that Pref-1 expression is less suppressed in IRS-1KO cells compared with WT cells. This may account for the high levels of Pref-1 in IRS-1KO cells leading to the defect in differentiation. Together, these data suggest a critical role of the promoter-proximal SBE9 in direct binding of Smad1/4 complex, which mediates BMP7's suppressive effect on Pref-1 gene expression.

### Example 6

### BMP7 Induces Spontaneous Differentiation in Wild Type Brown Preadipocytes and Rescues Brown Preadipogenesis in IRS-1 KO Cells

To further verify the findings that BMP7 rescued the differentiation defect of IRS-1KO cells, BMP7 was stably expressed in WT and IRS-1KO cells by transfecting these cells with a vector expressing human BMP7 (hBMP7) under the control of the adipocyte-specific aP2 promoter.

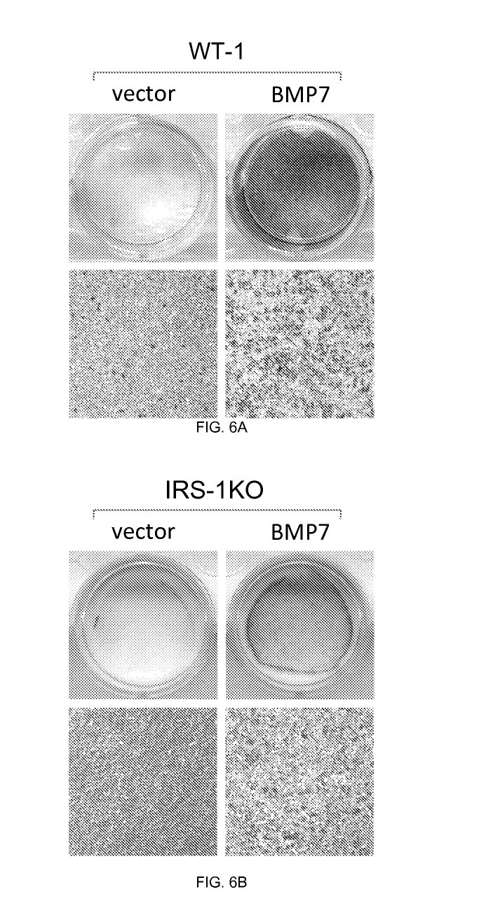

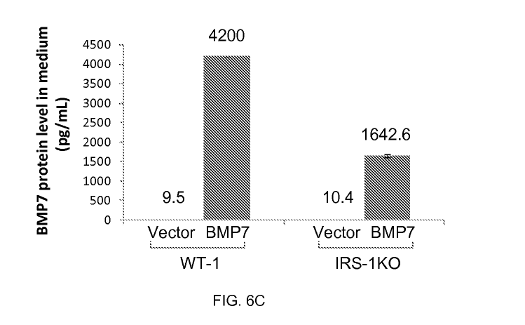

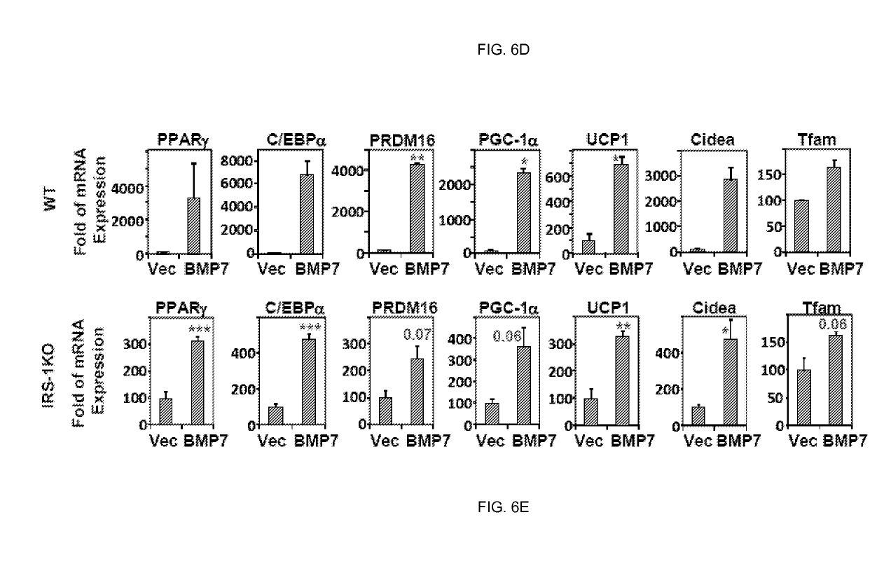

As shown in FIG. 6C, ELISA analysis confirmed high levels of hBMP7 in the culture media collected on day 10.

As shown in FIG. 6A, when subjected to continuous post-confluence culture, the WT cells overexpressing BMP7 underwent spontaneous brown adipocyte differentiation, as determined by lipid accumulation, and displayed increased expression of general and brown adipogenic marker genes (see FIG. 6D).

As shown in FIG. 6B, overexpression of BMP-7 in IRS-1KO cells did not result on spontaneous differentiation; however, when put in the standard differentiation media, the BMP7-overexpessing IRS-1KO cells were able to differentiate efficiently into mature brown adipocytes, which expressed high levels of the key adipogenic transcription factors PPARγ and C/EBPα, as well as brown fat-selective markers PRDM16, PGC-1α, Cidea and UCP1 (FIG. 6E). Given the fact that there is little to no aP2 expression in preadipocytes, it is conceivable that IRS-1KO cells transfected with the aP2 promoter-driven BMP7 vector still require induction cocktails to initiate differentiation, but once the cells enter the adipogenic program, they are able to produce BMP7 to further enhance differentiation.

These results support that BMP-7 promotes brown adipogenesis in Wt brown preadipocytes and rescues the differentiation defect in IRS1-KO cells.

To confirm the above results were truly BMP-7-dependent, BMP-7 signaling was inhibited. Briefly, WT-1 cells were treated with vehicle or 23 nM noggin, an antagonist of BMP, and subjected to MDI-induced adipocyte differentiation upon confluency (defined as Day 0). On day 6, mRNA were isolated for gene expression by qPCR and culture dishes were Oil Red O stained.

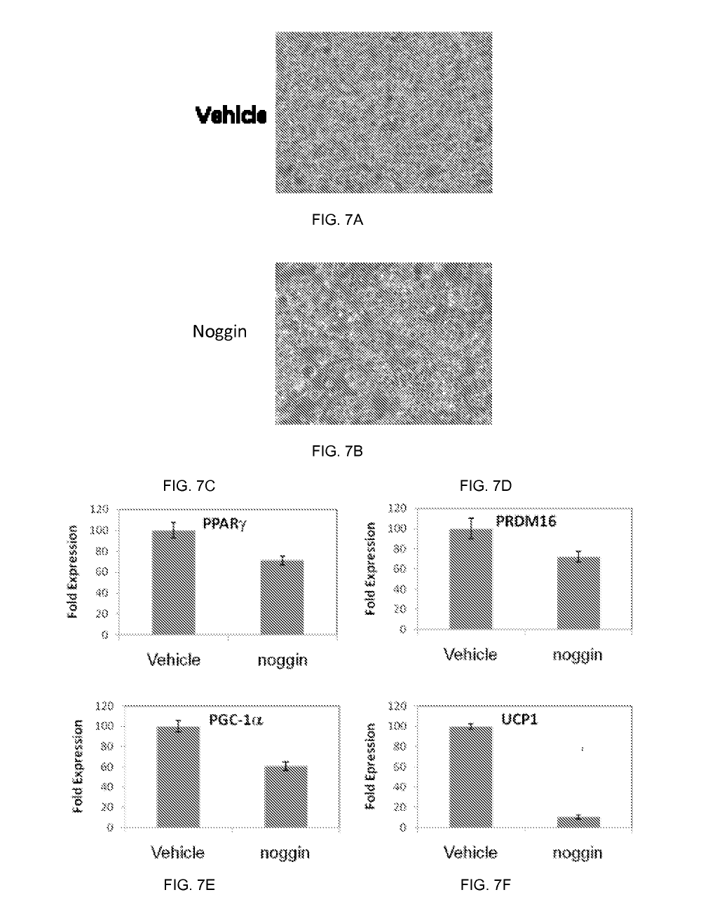

As shown in FIG. 7, noggin treatment impairs brown adipogenesis in Wt cells, suggesting that BMP signaling is required for initiating a full brown adipogenic program.

Together, the data presented above support cross-talk between BMP-7 and insulin signaling by which BMP-7 can rescue brown adipogenesis in cells with insulin resistance.

### Example 8

### BMP7 Treatment Reduces Insulin Resistance, Promotes Glucose Sensitivity and Protects Against States of Insulin Resistance (Fatty Liver) In Vivo

To determine if BMP7 is able to regulate insulin sensitivity in vivo, obese mice made by high fat diet were injected with adenoviruses expressing BMP7 or LacZ as a control. The obese mice were generated by feeding the C57BL/6 mice with a high fat/hypercaloric diet (45% calorie from fat) for 12 weeks. One week after adenoviral injection, the mice were subjected to glucose and insulin tolerance tests.

Glucose and insulin tolerance tests were performed as follows Animals were subjected to a 4-15 hour fasting period (with free access to water), whereafter either 2 g/kg BW of glucose or 0.75 iU/kg BW of insulin was injected intraperitoneally. Blood glucose concentrations were determined by the tail nick procedure. Blood was collected at 10 minutes pre injection and 15, 30, 60 and 120 minutes post injection. Mice were fed immediately after the last blood sample was collected. In order to reduce the numbers of animals used in experiments, these two tests were performed sequentially with a 1 2 weeks interval between tests.

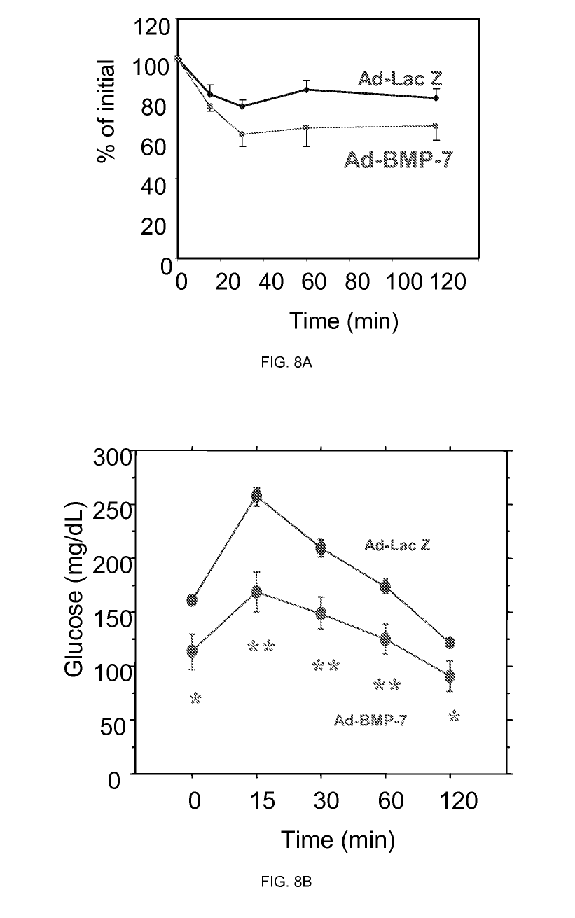

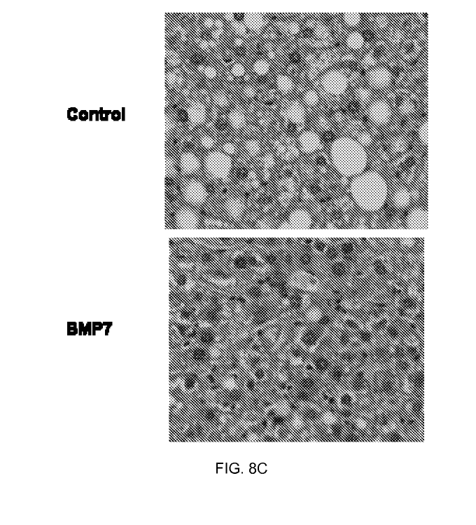

As shown in FIG. 8A, BMP7 improves insulin sensitivity compared to control (LacZ). BMP7 also improves glucose sensitivity in treated animals (FIG. 8B). As a result, BMP7 also reduces hepatic steatosis (FIG. 8C).

These results support that BMP7 can be used to treat insulin resistance in vivo. As shown herein, systemic administration of BMP7 in the well established diet-induced obesity (DIO) mouse model, which closely resembles obesity in humans (Augustine and Rossi, Anat. Rec., 257:64 72 (1999)), resulted in decreased insulin resistance, or heightened insulin sensitivity, in the mice.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a condition associated with insulin resistance in a subject, the method comprising selecting a subject with insulin resistance and administering a therapeutically effective amount of one or more BMP7 agents to the subject, thereby treating insulin resistance in the subject.

2. The method of claim 1, wherein selecting a subject with insulin resistance comprises selecting a subject with one or more of diabetes, pre-diabetes, obesity, hypertension, metabolic syndrome, polycystic ovary disease, and fatty liver.

3. The method of claim 1, wherein selecting a subject with insulin resistance comprises determining insulin resistance in the subject.

4. The method of claim 3, wherein insulin resistance is determined directly.

5. The method of claim 1, wherein the BMP7 agent is administered systemically.

6. The method of claim 1, wherein the BMP7 agent comprises a BMP7 polypeptide.

7. The method of claim 6, wherein the BMP7 polypeptide comprises a polypeptide with at least 95% identity to amino acids 293-431 of SEQ ID NO:5.

8. The method of claim 1, wherein the BMP7 agent comprises a BMP7 peptide or peptide mimetic.

9. The method of claim 1, wherein the one or more BMP7 agents comprises a nucleic acid molecule encoding a BMP7 polypeptide.

10. The method of claim 9, wherein the nucleic acid molecule encodes a BMP7 polypeptide with at least 95% identity to amino acids 293-431 of SEQ ID NO:5.

11. The method of claim 1, further comprising assessing the level of insulin resistance in the subject before and after administration of the BMP7 agent, wherein the level of insulin resistance before administration is greater than the level of insulin resistance after administration.


SEQUENCE LISTING (9 sequences)

SEQ ID NO: 1 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 2 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEAISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 3 - protein, 454 aa, Homo sapiens
MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 4 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 5 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 6 - protein, 16 aa, Artificial Sequence
  Description of Artificial Sequence Synthetic peptide
CIVNSSDDFLCKKYRS

SEQ ID NO: 7 - protein, 16 aa, Artificial Sequence
  Description of Artificial Sequence Synthetic peptide
CYFNDSSQVLCKRYRS

SEQ ID NO: 8 - DNA, 14 bp, Artificial Sequence
  Description of Artificial Sequence Synthetic oligonucleotide
cgtctagccaaaga

SEQ ID NO: 9 - DNA, 29 bp, Artificial Sequence
  Description of Artificial Sequence Synthetic oligonucleotide
tgtctgtctgtctgtctgtctgtctgcat